(19) United States
(12) Patent Application Publication
(10) Pub. No.: US 2022/0075419 A1
(43) Pub. Date: Mar. 10, 2022

(54) FLEXIBLE WIRING SUBSTRATE AND ELECTRONIC APPARATUS

(71) Applicant: SONY GROUP CORPORATION, TOKYO (JP)

(72) Inventor: AKIHIRO NOMOTO, TOKYO (JP)

(21) Appl. No.: 17/414,481

(22) PCT Filed: Oct. 31, 2019
(86) PCT No.: PCT/JP2019/042803

§ 371 Date: Jun. 16, 2021

(30) Foreign Application Priority Data
Dec. 26, 2018 (JP) 2018-242883

(51) Int. Cl. G06F 1/16; G06F 1/18

(52) U.S. Cl. CPC G06F 1/1652; A61B 5/0245; G06F 1/189; G06F 1/163

(57) ABSTRACT

[Object] To provide a flexible wiring substrate and an electronic apparatus that can easily provide the substrate with stretchability.

[Solving Means] A flexible wiring substrate includes a plurality of non-stretching portions and stretching portions. The plurality of the non-stretching portions is spaced apart from each other. The stretching portions divide the plurality of the non-stretching portions, connect the adjacent non-stretching portions, and are stretched and contracted by formed slits to be capable of changing relative positions between the non-stretching portions.

### TECHNICAL FIELD

The present technology relates to a flexible wiring substrate and an electronic apparatus.

### BACKGROUND ART

Patent Literature 1 discloses a biosensor device having high stretchability which can follow a movement of a living body. The biosensor device has non-stretching portions and stretching strips being connecting with the non-stretching portions. The stretching strips exhibit sufficient stretchability as they are stretched and contracted. Each stretching strip is configured to be curved in a serpentine shape or a spiral shape. A plurality of the non-stretching portions is spaced apart from one another, and each stretching strip is arranged between the adjacent non-stretching portions.

In the biosensor device described in Patent Literature 1, a wiring layer is formed in each stretching strip and each non-stretching portion. When the wiring layer is planarly viewed, there are regions in which the stretching strips exist and regions including no stretching strip from which the wiring layer is removed exist among the plurality of the non-stretching portions.

### CITATION LIST

### Patent Literature

Patent Literature 1: Japanese Patent Application Laid-open No. 2017-113088

### DISCLOSURE OF INVENTION

### Technical Problem

As the biosensor device described in Patent Literature 1, in a case where the stretching strip is formed such that the regions including no stretching strip exist between the adjacent non-stretching portions, it is necessary to remove the wiring layer in the regions including no stretching strip. Since the stretching strip is curved in the serpentine shape or the spiral shape, when the removed regions are fine, it is likely to occur a problem that the wiring layer to be removed is insufficiently removed.

In view of the above circumstances, it is an object of the present technology to provide a flexible wiring substrate and an electronic apparatus that can easily provide the substrate with stretchability.

### Solution to Problem

In order to achieve the above object, a flexible wiring substrate according to an embodiment of the present technology includes a plurality of non-stretching portions and stretching portions.

The plurality of the non-stretching portions is spaced apart from each other.

The stretching portions divide the plurality of the non-stretching portions, connect the adjacent non-stretching portions, and are stretched and contracted by formed slits to be capable of changing relative positions between the non-stretching portions.

According to such a configuration, it is possible to impart stretchability to the stretching portions by the formed slits, and obtain the flexible wiring substrate having the stretchability.

The slits may include first slits that separate the stretching portion located between two adjacent non-stretching portions from the stretching portion located between two other adjacent non-stretching portions.

The slits may include second slits each extending in non-parallel with an imaginary line connecting respective centers of the two adjacent non-stretching portions in the stretching portions located between the two adjacent non-stretching portions.

The second slits may be perpendicular to the imaginary line.

One end of the second slit may be connected to the first slit, and the other end may not be connected to the first slit.

A plurality of the first slits may be formed at the stretching portions, at least the two second slits may be formed between the two adjacent first slits, one end of one second slit of the two second slits may be connected to one of the two adjacent first slits, and one end of the other second slits may be connected to the other first slit.

The second slits may have widening portions at the other ends.

Reinforcing portions covering the other ends of the second slits may be further included.

The slits may have shapes extending in different directions.

The slits may be configured of linear portions.

Electronic components provided in the non-stretching portions and wiring provided in the stretching portions for electrically connecting the electronic components may be further included.

The flexible wiring substrate may be made of a conductive member.

In order to achieve the above object, an electronic apparatus according to an embodiment of the present technology may include flexible wiring substrate including a plurality of non-stretching portions spaced apart from each other and stretching portions that divide the plurality of the non-stretching portions, connect the adjacent non-stretching portions, and are stretched and contracted by formed slits to be capable of changing relative positions between the non-stretching portions.

### BRIEF DESCRIPTION OF DRAWINGS

FIG. 1 is a schematic plan diagram of a flexible wiring substrate according to a first embodiment of the present technology.

FIG. 2 is a diagram for describing shapes of slits formed in the flexible wiring substrate.

FIG. 3 is a partial enlarged diagram of FIG. 1, and is a diagram for showing a state in which a stretching portion is stretched.

FIG. 4 is a schematic diagram showing a configuration example of a heartbeat measuring apparatus according to a second embodiment of the present technology.

FIG. 5 is a schematic cross-sectional diagram of a sensor main body of the heartbeat measuring apparatus.

FIG. 6 is a schematic plan diagram of the flexible wiring substrate arranged on the heartbeat measuring apparatus.

FIG. 7 is a schematic plan diagram showing a configuration example of an LED display according to a third embodiment of the present technology.

FIG. 8 is a schematic plan diagram showing a configuration example of an array sensor according to a fourth embodiment of the present technology.

FIG. 9 is a schematic plan diagram showing other example of the flexible wiring substrate according to a fifth embodiment of the present technology.

FIG. 10 is a schematic plan diagram showing still other example of the flexible wiring substrate according to a sixth embodiment of the present technology.

FIG. 11 is a schematic plan diagram showing still other example of the flexible wiring substrate according to a seventh embodiment of the present technology.

FIG. 12 is a schematic plan diagram showing still other example of the flexible wiring substrate according to an eighth embodiment of the present technology.

FIG. 13 is a schematic plan diagram showing still other example of the flexible wiring substrate according to a ninth embodiment of the present technology.

FIG. 14 is a schematic plan diagram showing still other example of the flexible wiring substrate according to a tenth embodiment of the present technology.

FIG. 15 is a schematic plan diagram showing still other example of the flexible wiring substrate according to an eleventh embodiment of the present technology.

FIG. 16 is a schematic plan diagram showing still other example of the flexible wiring substrate according to a twelfth embodiment of the present technology.

FIG. 17 is a schematic plan diagram showing still other structural example of the flexible wiring substrate according to a thirteenth embodiment of the present technology.

### MODE(S) FOR CARRYING OUT THE INVENTION

Hereinafter, a flexible wiring substrate according to the present technology and an electronic apparatus using the same will be described. In the description, the same components are denoted by the same reference numerals, and the description of the components that have already been described is omitted in some cases.

<Schematic Configuration of Flexible Wiring Substrate>

The flexible wiring substrate according to the present technology has flexibility and stretchability. The flexible wiring substrate has non-stretching portions and stretching portions arranged so as to divide the non-stretching portions in a non-stretching state. The stretching portions and the non-stretching portions are integrally connected. The stretching portions exhibit the stretchability by forming slits. Thus, the flexible wiring substrate has the flexibility and the stretchability, whereby relative positions of the adjacent non-stretching portions can be changed.

In the non-stretching portions, electronic components, wiring, pad portions, electrodes, and the like may be arranged.

The wiring, electrodes, and the like may be arranged in the stretching portions.

The electronic components include, for example, an IC (integrated circuit) chip, an LED (light emitting diode), a photodiode, a transistor, various sensors, a solar cell, a resistor, a capacitor, a transformer, an inductor, and the like.

An existing conductive material may be used for the wiring, the pad portion, the electrode. For example, a single metal or an alloy of copper (Cu), platinum (Pt), titanium (Ti), ruthenium (Ru), molybdenum (Mo), tungsten (W), nickel (Ni), aluminum (Al), tantalum (Ta), or the like can be used.

In addition, a stretching conductive material such as silver nanowires may be used as the wiring material, and by forming the wiring in the stretching portions using such a material, it is possible to further suppress disconnection failure of the wiring due to deformation of the stretching portions.

The flexible wiring substrate has a form, for example, in which the electronic components and the like are provided in the non-stretching portions, and in which the wiring for electrically connecting the electronic components to the stretching portions is provided.

Furthermore, the flexible wiring substrate may be a form in which no electronic component is provided, and patterned wiring, the pad portions, the electrodes and the like may be provided.

Alternatively, the flexible wiring substrate itself may be configured of a conductive member and function as the electrode.

The flexible wiring substrate having the form in which the electronic components, the wiring, and the like are provided includes, for example, an insulating substrate including the formed slits, and the electronic components, the wiring, and the like arranged on the insulating substrate. The insulating substrate is a support member for supporting the electronic components and the wiring.

As the insulating substrate, a flexible substrate which is flexible and can be repeatedly deformed with a low force can be used. For example, a resin member such as polyimide and polyester, or an elastic member such as an elastomer can be used for the insulating substrate.

Furthermore, the flexible wiring substrate functioning as the electrode made of the conductive member has a configuration that the slits are formed in a single metal sheet made of a low conductive member resistance value such as copper, silver, and aluminum.

For example, the flexible substrate can be obtained by using the metal sheet having a thickness of 2 mm or less. By forming the slits in such a metal sheet, the flexible wiring substrate having the stretchability and the flexibility can be obtained.

Here, although the thickness is 2 mm or less, this is an example and is not limited to this numerical value, and it is possible to set the appropriate thickness to exhibit the flexibility considering a type, a size, and workability of the member used.

Furthermore, in still other embodiment, the flexible wiring substrate may be configured of a composite member including a support member and a conductive layer like a solid film formed on the support member and may have the slits in both the support member and the conductive layer. The conductive layer functions, for example, as an electrode.

The composite member may be configured by bonding the metal sheet to the support member, or may be formed by applying, printing, vapor-depositing, sputtering or the like a conductive material on the support member to form the conductive layer, and is not limited.

As the composite member, a flexible member which is flexible and can be repeatedly deformed with a low force can be used. As the support member, the above-mentioned insulating substrate can be used.

As to the flexible wiring substrate of the present technology, when a force is applied, the stretching portions are deformed, the relative positions of the non-stretching portions oppositely arranged through the stretching portions can be changed. Thus, it is possible to three-dimensionally deform the flexible wiring substrate.

Hereinafter, by way of specific embodiments, the flexible wiring substrate, and the electronic apparatus using the flexible wiring substrate will be described in detail. In each of the following drawings, three mutually perpendicular directions are referred to as the X direction, the Y direction, and the Z direction, respectively.

### First Embodiment

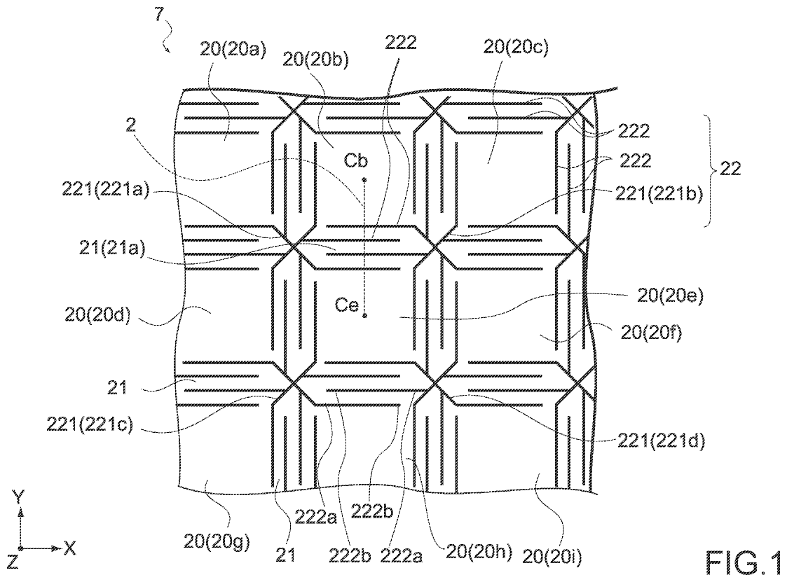

A configuration example of the flexible wiring substrate according to an embodiment of the present technology will be described with reference to FIGS. 1 to 3. In this embodiment, shapes of the slits mainly formed on a flexible wiring substrate 7 are described, and the wiring, the electronic components, and the like are therefore not shown in FIGS. 1 to 3.

FIG. 1 is a partial plan diagram of the flexible wiring substrate 7 according to this embodiment.

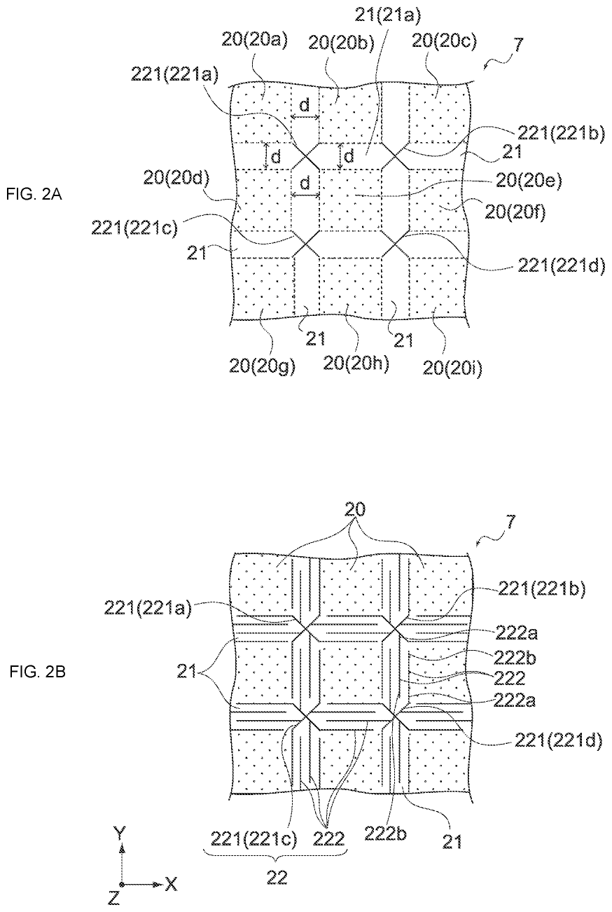

FIG. 2 is a diagram for describing shapes of the slits formed in the flexible wiring substrate 7 of FIG. 1. In FIG. 2, regions to be the non-stretching portions 20 are shown as being filled with dots. Furthermore, in FIG. 2(A), it shows contours of the regions to be non-stretching portions 20 by dotted lines.

As shown in FIGS. 1 and 2, the flexible wiring substrate 7 of this embodiment has the plurality of the non-stretching portions 20 and stretching portions 21 for dividing the non-stretching portions 20.

By forming the slits 22, the plurality of the non-stretching portions 20 having substantially square shapes and the stretching portions 21 having the stretchability are formed. The non-stretching portions 20 and the stretching portions 21 are connected.

The plurality of the non-stretching portions 20 is arranged apart from each other. Peripheries of the non-stretching portions 20 are surrounded by the stretching portions 21. In FIGS. 1 and 2, reference numerals 20a to 20i are used to distinguish the respective plurality of the non-stretching portions 20, but if it is not necessary to distinguish among them, it will be described as the non-stretching portions 20.

In the non-stretching portions 20, the electronic components, the wiring, the pad portions, the electrodes and the like can be arranged.

The stretching portions 21 have lattice shapes along the X-axis and Y-axis perpendicular to each other in a non-stretching state.

By forming the slits 22, the stretchability is imparted to the stretching portions 21, and it is possible to be capable of changing the relative positions of the adjacent non-stretching portions 20. For example, it is possible to be capable of changing distances d between the adjacent non-stretching portions 20.

The slits 22 include first slits 221 and second slits 222. The slits 22 are configured of a plurality of linear portions.

The slits 22 have shapes configured of the linear portions extending in different directions from each other. The second slits 222 include slits respectively extending along the X-axis direction and the Y-axis directions different each other. Thus, it is possible to stretch and contract the flexible wiring substrate 7 in multiple directions.

FIG. 2(A) is a diagram for describing the positional relationships between the first slits 221 and the non-stretching portions 20, and the second slits 222 are not shown. In FIG. 2(B), it shows both the first slits 221 and the second slits 222.

As shown in FIGS. 1 and 2(A), the first slits 221 have X-shapes and are formed at positions corresponding to intersections of regions of the lattice-shaped stretching portions 21.

Each of the first slits 221 is formed so that the stretching portion 21 located between two adjacent non-stretching portions 20 and the stretching portion 21 located between the other two adjacent non-stretching portions 20 are separated from each other. The first slits 221 are formed so as to connect the two adjacent non-stretching portions 20.

In this embodiment, the first slits 221 are arranged so as to connect the two adjacent non-stretching portions 20 in the lateral direction (X-axis direction), the two adjacent non-stretching portions 20 in the longitudinal direction (Y-axis direction), and the two adjacent non-stretching portions 20 in the oblique direction, respectively.

In FIG. 1 and FIG. 2, reference numerals 221a to 221d are used to distinguish the plurality of the first slits 221 from each other for the sake of description, but when there is no particular need to distinguish them, they will be described as the first slits 221.

In FIG. 1, for example, one of the two mutually intersecting linear portions constituting the first slit 221a is formed so as to connect the non-stretching portions 20b and 20d adjacent to each other in the oblique direction, and the other is formed so as to connect the non-stretching portions 20a and 20e adjacent to each other in the oblique direction.

Thus, the first slit 221a connects the two non-stretching portions 20a and 20b, 20d and 20e adjacent in the lateral direction, the two non-stretching portions 20a and 20d, 20b and 20e adjacent in the longitudinal direction, and the two non-stretching portions 20a and 20e, 20b and 20d adjacent in the oblique direction.

The same applies to the other first slits 221b to 221d.

By forming the first slit 221, for example, the stretching portion 21 located between the two adjacent non-stretching portions 20a and 20b, the stretching portion 21 located between the other two adjacent non-stretching portions 20d and 20e, the stretching portion 21 located between the non-stretching portions 20a and 20d, and the stretching portion 21 located between the non-stretching portions 20b and 20e are not connected and separated from each other.

As shown in FIGS. 1 and 2(B), the plurality, four in this embodiment, of the second slits 222 is formed between the two adjacent non-stretching portions 20 in the X-axis direction (lateral direction) or the Y-axis direction (longitudinal direction), i.e., between the two adjacent first slits 221.

The second slits 222 extend in non-parallel with an imaginary line 2 connecting between centers C of the two adjacent non-stretching portions 20, e.g., perpendicular to the imaginary line 2 in this embodiment. The second slits 222 have a shape in which there is no connection between the two adjacent non-stretching portions 20.

Thus, by extending the second slits 222 in non-parallel with the imaginary line 2, it is possible to efficiently widen the distances d between the non-stretching portions 20 oppositely arranged through the second slits 222. Furthermore, by extending the second slits 222 perpendicular to the imaginary line 2, it is possible to more efficiently widen the distances d between the non-stretching portions 20.

Here, perpendicular includes a range of the angle formed between the second slit 222 and the imaginary line 2 of 85° to 90°.

In FIG. 1, for example, four second slits 222 extending in the X-axis direction are formed in the stretching portion 21a located between the non-stretching portions 20b and 20e. These second slits 222 are perpendicular to the imaginary line 2 connecting respective centers Cb, Ce of the non-stretching portions 20b and 20e.

The four second slits 222 are formed in parallel with each other.

Parts of the second slits 222 are formed so as to form contours of substantially square shapes of the non-stretching portion 20, while leaving parts where the non-stretching portions 20 and the stretching portions 21 are connected.

Each second slit 222 includes one end 222a and the other end 222b. The one end 222a is connected to the first slit 221. The other end is not connected to the first slit 221.

In FIG. 1, the four second slits 222 in parallel with each other forming the stretching portion 21a located between the two adjacent first slits 221a and 221b are arranged such that, in order from top to bottom, the second slit 222 for connecting to the first slit 221b, the second slit 222 for connecting to the first slit 221a, the second slit 222 for connecting to the first slit 221b, and the second slit 222 for connecting to the first slit 221a.

That is, the second slit 222 connected to the first slit 221a and the second slit 222 connected to the first slit 221b are alternately formed.

Thus, as to the plurality of the second slits 222 forming the stretching portions 21 located between the two adjacent first slits 221 in the lateral or longitudinal direction, the second slit 222 for connecting one of the first slits 221 of the two adjacent first slits 221 and the second slit 222 for connecting the other first slit 221 are formed alternately.

Thus, the second slits 222 are formed in respective divided regions provided by dividing the regions corresponding to the stretching portions 21 into a plurality of divided regions by the first slits 221, thereby forming the stretching portions 21 each having a form of an elongated strip-shaped body.

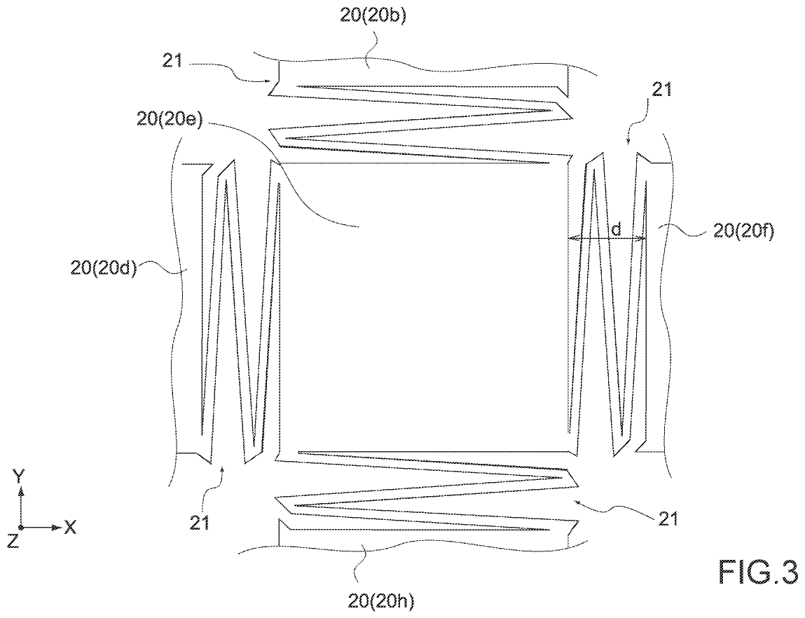

Hereinafter, description will be made with reference to FIGS. 2 and 3.

FIG. 3 is a partial enlarged diagram of FIG. 1, and is a schematic diagram for describing a state in which the stretching portion 21 is stretched.

As shown in FIG. 2(A), the regions corresponding to the stretching portions 21 are divided into the plurality of regions in the X-axis direction and the Y-axis direction by the plurality of the first slits 221. The divided regions are connected only to the two non-stretching portions 20 oppositely arranged through the divided regions.

Furthermore, as shown in FIGS. 2(B) and 3, by forming the second slits 222, each divided region has the form of the elongated strip-shaped body in a zigzag shape in which Z-shaped linear lines are folded back many times. As a result, the plurality of the non-stretching portions 20 has shapes connected to each other through stretching portions 21 each having the strip-shaped body in the zigzag shape being stretchable and contractable.

By forming the strip-shaped body in the zigzag shape, the stretching portions 21 exhibit the stretchability.

That is, in this embodiment, as shown in FIG. 1, in a non-stretching state where no stress is applied to the flexible wiring substrate 7, no gap is generated between the non-stretching portions 20 such that the substrate is partially penetrated.

However, in a state that the flexible wiring substrate 7 is bent under stress, for example, as shown in FIG. 3, the stretching portions 21 of the strip-shaped bodies in the zigzag shapes are stretched. As described above, the stretching portions 21 exhibit the stretchability by forming the slits 22. By stretching the stretching portions 21 of the strip-shaped bodies in the zigzag shapes, the gaps are generated in the stretching portions 21, and the distances d between the non-stretching portions 20 are changed.

Thus, in the flexible wiring substrate 7, one stretching portion 21 of the strip-shaped body in the zigzag shape is connected to each of the two non-stretching portions 20 which are oppositely arranged through the stretching portion 21, but is not connected to other non-stretching portions 20.

Therefore, a deformation of one stretching portion 21 of the strip-shaped body in the zigzag shape is difficult to follow a deformation of the stretching portion 21 of the other strip-shaped body, and it is less likely to be affected.

Furthermore, since the stretching portions 21 are configured to be stretchable, even when the stress is applied to the flexible wiring substrate 7, the stress is easily dispersed in the stretching portions 21. Therefore, a positional change of one of the two non-stretching portions 20 connected via one stretching portion 21 of the strip-shaped body is less likely to affect a positional change of the other non-stretching portion 20. Therefore, an arrangement position of each non-stretching portion 20 can be changed almost independently.

Accordingly, it is possible to flexibly deform the flexible wiring substrate 7 three-dimensionally.

In this embodiment, in a state where no force is applied to the flexible wiring substrate 7, since there is no gap in the flexible wiring substrate 7, it is possible to set short distances d between the non-stretching portions 20. Therefore, it is possible to shorten arrangement pitches of arrangeable electronic components in the non-stretching portions 20, such that it is possible to increase the density of the arrangement of the electronic components and to widen a design range of the flexible wiring substrate.

Here, in the biosensor device described in Patent Literature 1 described above, there are the region where the stretching strips exist and the region where no stretching strip exists between the adjacent non-stretching portions when planarly viewed. The region where no stretching strip exits is the region where the wiring layer is penetrated and removed.

Thus, in order to form the stretching strip curved in a serpentine shape or a spiral shape formed by partially penetrating and removing the wiring layer, it is necessary to provide the region where the wiring layer is removed. For this reason, in the biosensor device described in Patent Literature 1, it is difficult to set the distances between the non-stretching portions determined by the length of the stretching strips at the time of non-stretching to be short, and it is difficult to increase the density of the arrangement of the electronic components.

In contrast, in this embodiment, since it is possible to impart the stretchability to the substrate only by forming the slits in the substrate and it is not necessary to penetrate and remove the substrate, it is possible to set short distances between the adjacent non-stretching portions 20. Therefore, it is possible to shorten the distances between electronic components capable of being arranged on the non-stretching portions 20, and it is possible to arrange a plurality of electronic components on the substrate at a high density.

The slits 22 can be formed by, for example, laser cutting. In this embodiment, since it is not necessary to laser cut the substrate into a partially penetrating shape, a processing apparatus having a low resolution forms easily the slits.

Here, if the stretching strip curved in a serpentine shape or a spiral shape described in Patent Literature 1 is formed by laser cutting, a material removing step of penetrated portions is required, and the number of steps increases. In addition, if a size of the removed portions is fine, defects may be generated such that any portion cannot be completely removed or the stretching strips are caught by something.

In contrast, in this embodiment, since it is not necessary to form the partially penetrating shape, the step of removing the material of the penetrated portions becomes unnecessary. Therefore, the number of processes can be reduced and a yield can be improved.

Furthermore, in this embodiment, since the shape of the slits 22 is constituted by a plurality of simple linear portions, for example, the processing apparatus having a low resolution forms easily the slits, and the flexible wiring substrate having a stable quality can be stably provided.

Furthermore, since the pattern of each slit 22 is constituted of a simple linear portion, it is possible to increase a processing speed.

In addition, since the shape of each second slit 222 is constituted of a simple line, it is easy to adjust the number of slits 22 formed in the stretching portions 21.

For example, it is easy to form a larger number of the second slits 222 in the stretching portions 21, and it is possible to form the stretching portions 21 each having a long strip-shaped body including a large number of folds. As a result, it is possible to improve the stretchability of the flexible wiring substrate 7.

<Electronic Apparatus>

In the following second to fourth embodiments, an electronic apparatus including the flexible wiring substrate according to an embodiment of the present technology will be described.

In the second to fourth embodiments, an example will be described in which a flexible wiring substrate including an insulating substrate including slits and electronic components, wiring, and the like arranged on the insulating substrate is applied.

### Second Embodiment

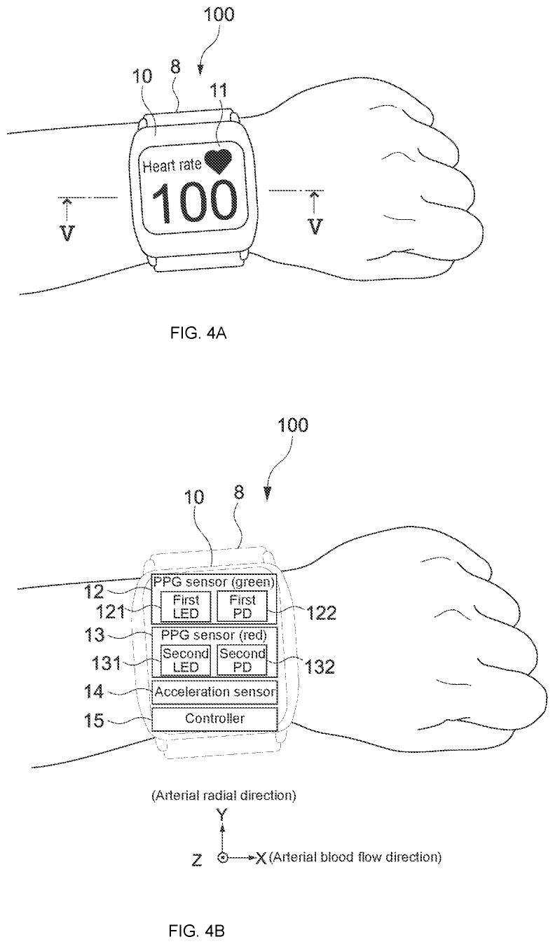

A heartbeat measuring apparatus as an example of the electronic apparatus will be described with reference to FIGS. 4 to 6.

[Schematic Configuration of Heart Rate Measurement Apparatus]

FIG. 4(A) is a schematic diagram showing a configuration example of a heartbeat measuring apparatus 100, and FIG. 4(B) is a schematic cross-sectional diagram showing a schematic configuration of the heartbeat measuring apparatus 100.

The heartbeat measuring apparatus 100 is a wristband-type photoplethysmography (PPG) heartbeat sensor and is used by being worn on a wrist of a user. The heartbeat measuring apparatus 100 is a biological information processing apparatus.

A PPG method is a method of measuring a pulse wave by a volume variation of a blood flow. In the PPG method, a light beam is irradiated from a light emitting unit such as an LED (light emitting diode) toward a skin. The irradiated light beam is absorbed, scattered, or reflected by blood and a subcutaneous tissue existing under a skin in the order of several mm. At this time, an amount of light returned from beneath the skin is measured, for example, by a light receiving unit such as a PD (photo diode) to measure a change in the blood flow of capillaries distributed under the skin.

As shown in FIG. 4, the heartbeat measuring apparatus 100 includes a wearing band 8 and a sensor body 10. The wearing band 8 is connected to the sensor body 10, and is in contact with and holds the wrist of the user. The specific configuration of the wearing band 8 is not limited.

The sensor body 10 has a display unit 11 on which a measured heart rate is displayed. The display unit 11 is a display device using, for example, liquid crystal or electroluminescence (EL). A touch panel may be configured as the display unit 11, and a user operation may be input.

As shown in FIG. 4(B), the sensor body 10 includes a first PPG sensor 12, a second PPG sensor 13, an acceleration sensor 14, and a controller 15. The first PPG sensor 12, the second PPG sensor 13, the acceleration sensor 14, and the controller 15 are provided in a flexible wiring substrate 17 to be described later.

The first and second PPG sensors 12 and 13 are provided on a surface side in contact with the wrist of the user. The acceleration sensor 14 and the controller 15 may be provided on a surface side in contact with the wrist of the user, or may be provided on an opposite surface side thereof.

The first PPG sensor 12 includes a first LED (light emitting diode) 121, which is a first light emitting unit, and a first PD (photo diode) 122, which is a first light receiving unit.

The first LED 121 emits green light in a green wavelength range (e.g., from about 500 nm to about 570 nm) toward a measurement site as light in the first wavelength range.

The first PD 122 detects an amount of green light reflected back from beneath the skin of the measurement site.

The first PPG sensor 12 is provided mainly for measuring a change in the blood flow.

The second PPG sensor 13 includes a second LED 131, which is a second light emitting unit, and a second PD 132, which is a second light receiving unit.

The second LED 131 emits red light in a red wavelength range (e.g., from about 620 nm to about 750 nm) toward the measurement site as light in the second wavelength range.

The second PD 132 detects an amount of reflected light of red light returned from beneath the skin of the measurement site.

A long wavelength red light emitted from the second PPG sensor 13 reaches deep into a body tissue beneath the skin. Therefore, the red light emitted from the second PPG sensor modulates a return light due to deformation of the body tissue accompanied by, for example, a movement of fingers and wrist (movement of bones). Focusing on this point, in this embodiment, the second PPG sensor 13 is provided to generate a reference signal which is highly correlated with noise mainly caused by the movement of fingers and wrist.

In this embodiment, the first and second PPG sensors 12 and 13 constitute a pulse wave sensor unit. The first PPG sensor 12 functions as a pulse wave sensor for a pulse wave signal and generates the pulse wave signal.

The acceleration sensor 14 measures acceleration in the three axes XYZ of the measurement site to which the heartbeat measurement apparatus 100 is attached. The acceleration sensor 14 is provided for measuring a periodic movement of an arm, primarily when walking, jogging, running, or the like is performed. The acceleration sensor 14 functions as a body movement sensor, and the acceleration of each axis to be measured is output as a body movement signal. The specific configuration of the acceleration sensor 14 is not limited. As the body movement sensor, a three-axis gyro sensor or the like may be used in place of or in addition to the acceleration sensor 14.

In this embodiment, the lateral direction of the sensor body portion 10 is set as the X-axis direction, the longitudinal direction is set as the Y-axis direction. The direction perpendicular to each of the X-axis direction and Y-axis direction (perpendicular direction of surface of sensor body portion 10) is set as the Z-axis direction. In addition, the X-axis direction is regarded as an arterial blood flow direction of the measurement site, and the Y-axis direction is regarded as an arterial radial direction. Of course, it is not limited thereto.

The controller 15 controls operations of the first PPG sensor 12 and the second PPG sensor 13. Furthermore, the controller 15 generates heartbeat information of the user using the pulse wave signal from the first PPG sensor 12, a reference pulse wave signal from the second PPG sensor 13, and a body motion signal from the acceleration sensor 14.

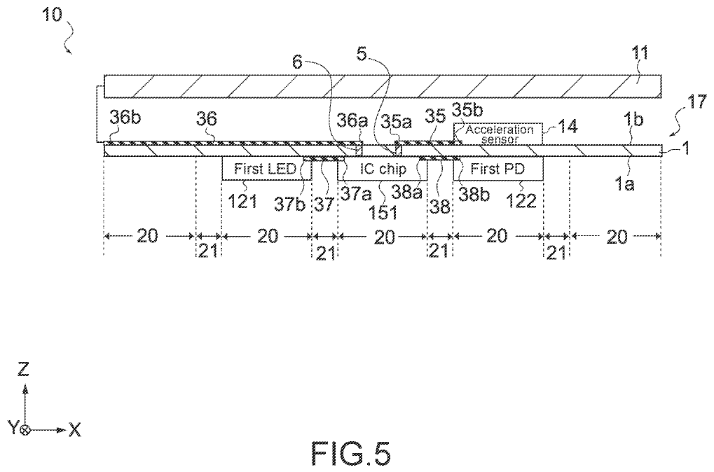

The controller 15 includes, for example, an IC chip 151 shown in FIG. 5.

FIG. 5 is a schematic cross-sectional diagram of the sensor main body of the heartbeat measuring apparatus taken along the line V-V in FIG. 4(A). FIG. 5 schematically shows a connection relationship between each electronic component and the wiring. In FIG. 5, the slits 22 are not shown.

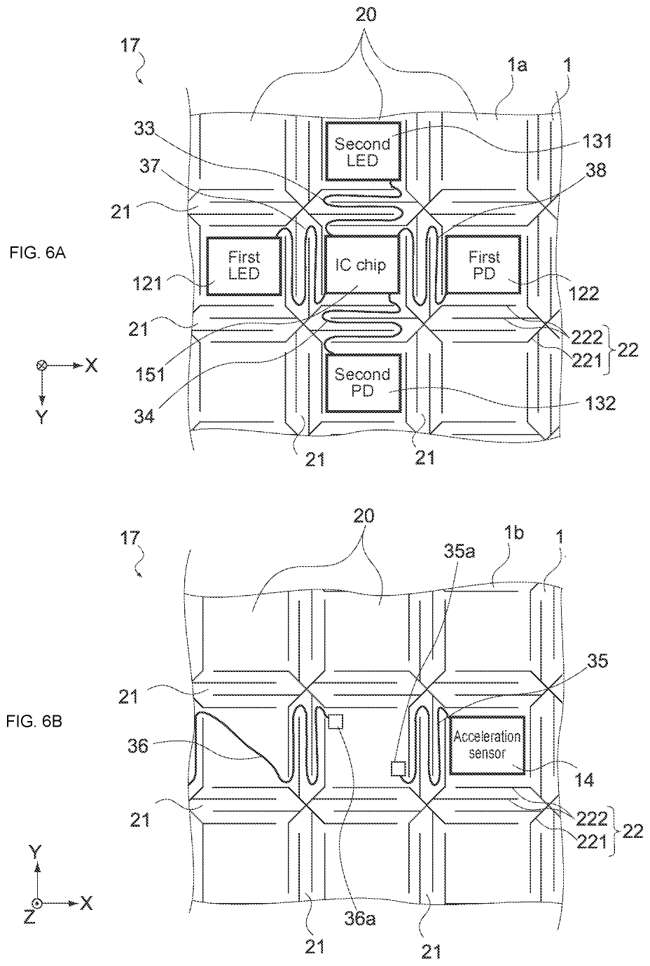

FIG. 6 is a partial schematic plan diagram of the flexible wiring substrate 17 constituting a part of the heartbeat measuring apparatus 100. FIG. 6(A) is a partial schematic plan diagram when viewed from one surface 1a of the flexible wiring substrate. FIG. 6(B) is a partial schematic plan diagram when viewed from the other surface 1b of the flexible wiring substrate.

As shown in FIG. 5, the sensor body 10 includes the display unit 11 and the flexible wiring substrate 17.

As shown in FIG. 6, the flexible wiring substrate 17 includes the plurality of the non-stretching portions 20 of the square shape, and the stretching portions 21 for dividing the non-stretching portions 20 similar to the flexible wiring substrate 7 of the first embodiment. Furthermore, in the flexible wiring substrate 17, the slits 22 having the same shapes as the flexible wiring substrate 7 described above are formed, and the stretching portions 21 are stretchable. The shapes of the slits 22 and the operation and effect of providing the slits 22 are the same as those of the first embodiment.

As shown in FIGS. 5 and 6, the flexible wiring substrate 17 includes the insulating substrate 1 in which the slits 22 are formed, the wiring 33 to 38, each first LED 121 as the electronic component, each first PD 122, each second LED 131, each second PD 132, each IC chip 151 constituting each controller 15, and each acceleration sensor 14.

The insulating substrate 1 has one surface 1a and the other surface 1b facing each other. One surface 1a is a surface located on a skin side of the user when the user wears the heartbeat measurement apparatus 100. The other surface 1b is a surface located on a display unit 11 side.

As shown in FIGS. 5 and 6(A), in the flexible wiring substrate 17, on one surface 1a of the insulating substrate 1, each first LED 121, each first PD 122, each second LED 131, each second PD 132, each IC chip 151, the wiring 33, 34, 37, 38 are arranged.

The first LED 121, the first PD 122, the second LED 131, the second PD 132, the IC chip 151, the pad portions arranged at both end portions of the wiring 33 (not shown), the pad portions arranged at each end portion of the wiring 34 (not shown), the pad portions 37a and 37b arranged at each end portion of the wiring 37, the pad portions 38a and 38b arranged at each end portion of the wiring 38 are respectively arranged in the non-stretching portions 20.

The wiring 37 electrically connects the first LED 121 and the IC chip 151.

The pad portion 37a of one end of the wiring 37 is electrically connected to the IC chip 151, the pad portion 37b of the other end is electrically connected to the first LED 121.

The wiring 37 is arranged in each stretching portion 21 located between each non-stretching portion 20 in which the first LED 121 is arranged and each non-stretching portion 20 in which the IC chip 151 is arranged.

The wiring 38 electrically connects the IC-chip 151 and the first PD 122.

The pad portion 38a of one end of the wiring 38 is electrically connected to the IC chip 151, the pad portion 38b of the other end is electrically connected to the first PD 122.

The wiring 38 is arranged in each stretching portion 21 located between each non-stretching portion 20 in which the IC chip 151 is arranged and each non-stretching portion 20 in which the first PD 122 is arranged.

The wiring 33 electrically connects the second LED 131 and the IC chip 151.

The pad portion at one end of the wiring 33 electrically connects to the IC chip 151, and the pad portion at the other end electrically connects to the second LED 131.

The wiring 33 is arranged in each stretching portion 21 located between each non-stretching portion 20 in which the second LED 131 is arranged and each non-stretching portion 20 in which the IC chip 151 is arranged.

The wiring 34 electrically connects the IC chip 151 and the second PD 132.

The pad portion at one end of the wiring 34 electrically connects to the IC chip 151, and the pad portion at the other end electrically connects to the second PD 132.

The wiring 34 is arranged in each stretching portion 21 located between each non-stretching portion 20 in which each IC chip 151 is arranged and each non-stretching portion 20 in which each second PD 132 is arranged.

As shown in FIG. 6(A), any of the wiring 33, 34, 37, 38 has a shape that is folded back a plurality of times along the zigzag shape of the belt-shaped body of the stretching portion 21. Therefore, a total length of each of the wiring 33, 34, 37, 38 is longer than a distance between the adjacent non-stretching portions 20.

As shown in FIGS. 5 and 6(B), in the flexible wiring substrate 17, in the other surface 1b of the insulating substrate 1, each acceleration sensor 14 and the wiring 35 and 36 are arranged.

Each acceleration sensor 14, the pad portions 35a and 35b which are arranged at both end portions of the wiring 35, the pad portions 36a and 36b which are arranged at both end portions of the wiring 36 are arranged in the non-stretching portions 20.

The wiring 35 electrically connects the IC chip 151 and the acceleration sensor 14.

The pad portion 35a of one end of the wiring 35 is electrically connected to the IC chip 151 via a through-hole via 5 formed in the insulating substrate 1, penetrated in the Z-axis direction, and copper-plated inside.

The pad portion 35b of the other end of the wiring 35 is electrically connected to the acceleration sensor 14.

The through-hole via 5 is formed in the non-stretching portion 20 in which the IC chip 151 is arranged.

The wiring 35 is arranged in each stretching portion 21 between each non-stretching portion 20 of the other surface 1b side where the IC chip 151 is arranged and each non-stretching portion 20 where the acceleration sensor 14 is arranged.

The wiring 36 electrically connects the IC chip 151 and the display unit 11.

The pad portion 36a of one end of the wiring 36 is electrically connected to the IC chip 151 via a through-hole via 6 formed in the insulating substrate 1, penetrated in the Z-axis direction, and copper-plated inside.

The wiring 36 is routed to a peripheral portion of the insulating substrate 1, and the pad portion 36b located at the other end of the wiring 36 is electrically connected to the display unit 11.

The through-hole via 6 is formed in the non-stretching portion 20 in which the IC chip 151 is arranged.

The wiring 36 is arranged in each stretching portion 21 and each non-stretching portion 20 passing from each non-stretching portion 20 of the other surface 1b side where the IC chip 151 is arranged to the peripheral portion of the insulating substrate 1.

As shown in FIG. 6(B), the wiring 35 and 36 arranged in each stretching portion 21 are arranged on the insulating substrate 1 along the zigzag shape of the strip-shaped body of each stretching portion 21 and has a shape that is folded back a plurality of times.

In this embodiment, for example, packaged electronic components are mounted on the insulating substrate 1 to form the flexible wiring substrate 7.

For example, a packaged LED includes a package substrate, LED elements, bonding wires, electrodes, and a sealing resin.

The package substrate is a substrate that serves as a base for the package.

The LED elements are provided on a package substrate.

The electrodes are metal terminals for flowing electricity and are provided on the substrate.

The bonding wires connect the LED elements and the electrodes of the substrate.

The sealing resin is provided so as to cover the LED elements in order to protect the LED elements from moisture and the like.

A known material, for example, copper or the like, is used for the wiring 33 to 38. Furthermore, it is desirable that the wiring 33 to 38 has a thickness such that the flexibility of the flexible wiring substrate 17 in the stretching portions 21 is not impaired when arranged in the stretching portions 21.

The flexible wiring substrate 17 is produced, for example, as follows.

In a base material including a pair of copper films on both surfaces of the insulating substrate 1, through holes are provided and are filled with a conductive material by electroplating or the like to form through-hole vias 5 and 6.

Next, the copper film is patterned by a photolithography process for each side to form wiring 33 to 38 including the pad portions.

Next, the electronic components such as the first LED 121, the first PD 122, the second LED 131, the second PD 132, the IC chip 151, the acceleration sensor 14 are connected to the corresponding pad portions using an anisotropic conductive film or solder or the like, and are mounted on the insulating substrate 1.

Thereafter, by forming the slits 22 in the insulating substrate 1 by laser cutting, to produce the flexible wiring substrate 17.

Note that the production method described here is an example, and is not limited thereto.

For example, after the formation of the wiring 33 to 38 and before mounting the electronic components, the slits 22 may be formed in the insulating substrate 1 by laser cutting.

In this embodiment, an example of mounting packaged electronic components is described, but it is not limited thereto. For example, the electronic components may be directly formed on the insulating substrate that is the support member, such as forming the LED elements directly on the insulating substrate 1 without mounting the LED package, electrically connecting the LED elements and the wiring, and covering the LED elements with the sealing resin.

Thus, the electronic components may be directly formed on the insulating substrate 1 to form the slits to produce the flexible wiring substrate.

Note that, in the following other embodiments, as the flexible wiring substrate for the electronic apparatus, a mode in which the packaged electronic components are mounted will be described as an example. Alternatively, the flexible wiring substrate may be produced by directly forming the electronic components on the substrate.

While this embodiment describes the example that the electronic components are located on both sides of the insulating substrate 1, the electronic components may be located only on one side.

Furthermore, for example, the flexible wiring substrate may be a multilayer substrate including the electronic components, i.e., the electronic components, the first wiring layer for connecting thereto, the insulating layer in which the through holes are formed, the second wiring layer for connecting to the electronic components via the through holes, and the cover insulating layer are sequentially stacked on the insulating substrate.

For example, in a case where the wiring for connection between the IC chips of each of the two different electronic components electrically connected to the IC chips is provided in the stretching portions 21 of the same region, the wiring may be formed such that each of the wiring is formed in the same layer and is not to electrically connected.

Alternatively, each of the wiring may be located in different layers through an interlayer insulating layer. In a case where the interlayer insulating layer is formed, the slits 22 are formed so as to penetrate the flexible wiring substrate including the interlayer insulating layer in the thickness direction. In this manner, a multilayer structure may be employed.

In the flexible wiring substrate 17 of this embodiment, by forming the slits 22, similar to the first embodiment, the stretching portion 21 is stretchable and the flexible wiring substrate 17 is three-dimensionally flexibly deformable.

By using the flexible wiring substrate 17 being deformable three-dimensionally in this manner, when the user wears the heartbeat measuring apparatus 100 on the wrist, the flexible wiring substrate 17 contacts the skin of the user and is easily deformed so as to conform to a surface shape of the skin.

As a result, presence of the gap between the flexible wiring substrate 17 and the skin can be reduced, and leakage of a light beam irradiated from the LED toward the skin due to the presence of the gap is reduced during the operation of the PPG sensor, thereby improving light utilization efficiency. In addition, light returning from beneath the skin can be efficiently detected.

Therefore, the change in the blood flow of capillaries distributed under the skin can be measured with high accuracy.

Furthermore, since the flexible wiring substrate 17 is easily deformed along the skin of the user, when wearing, it less likely to give a restraint feeling or discomfort of the body to the user, and misalignment due to the movement of the user is less likely to occur.

Furthermore, in this embodiment, when a force is applied to the flexible wiring substrate 17 upon bending, the stress is easily dispersed in the stretching portions 21 and is less likely to be applied to the non-stretching portions 20 where the electronic components are mounted. As a result, an occurrence of breakage of the electronic components arranged in the non-stretching portions 20 is suppressed, and the heartbeat measurement apparatus 100 with high reliability, in which electrical characteristics are maintained even by repeated wearing, can be obtained.

As described above, in this embodiment, the use of the flexible wiring substrate 17 which has the stretchability and can be deformed following a free-form curved surface enables the heartbeat measurement apparatus having high accuracy and high reliability.

While this embodiment describes the example in which it is applied to a wrist band type biological information processing apparatus which is a wearable apparatus worn on a wrist, but it is not limited thereto. Since the flexible wiring substrate 17 is three-dimensionally deformable, it can be worn on various body parts other than the wrist. For example, it can be applied to various wearable apparatuses such as a headband type, a neckband type, and a belt type, and by arranging the flexible wiring substrate in a shape along the body parts of the user, it is possible to detect biological information with high accuracy.

### Third Embodiment

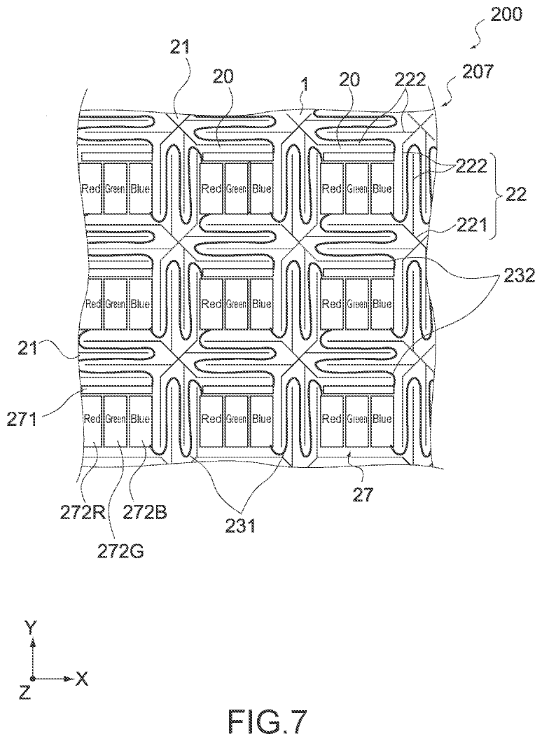

An LED display as an example of the electronic apparatus will be described with reference to FIG. 7.

FIG. 7 is a schematic plan diagram showing a configuration example of the LED display.

As shown in FIG. 7, an LED display 200 includes a flexible wiring substrate 207. The flexible wiring substrate 207 includes the insulating substrate 1 having the slits 22 formed therein, a plurality of LED packages 27 arranged on the insulating substrate 1, a plurality of data signal lines 232, and a plurality of address signal lines 231.

The plurality of the LED packages 27 is arranged in a matrix along the X-axis direction and the Y-axis direction perpendicular to each other.

The LED package 27, which is the electronic component, is arranged in the non-stretching portions 20 of the insulating substrate 1.

The LED package 27 has a red LED element 272R, a green LED element 272G, a blue LED element 272B, and three switching elements 271 provided for each LED element for driving each LED element.

Although the switching elements for the respective LED elements of respective colors are provided, as shown schematically in the drawing, a reference numeral 271 is applied to the three switching elements.

One pixel is constituted by three LED elements of a red LED element 272R, a green LED element 272G, and a blue LED element 272B.

The plurality of the data signal lines 232 is arranged along the Y-axis direction. The plurality of the address signal lines 231 is arranged along the X-axis direction. The data signal lines 232 and the address signal lines 231 are arranged intersecting each other via the interlayer insulating layer (not shown).

The data signal lines 232 and the address signal lines 231 are arranged in the stretching portions 21 and the non-stretching portions 20 of the insulating substrate 1, each portion arranged in the stretching portions 21 has a shape that is folded back a plurality of times along the zigzag shape of the belt-shaped body of the stretching portions 21.

The switching elements such as thin film transistors and the LED elements connected to the switching elements are provided at respective intersection of the data signal lines 232 and the address signal lines 231.

Each switching element includes, for example, a gate electrode, a gate insulating film, a semiconductor layer forming a channel region, and a pair of source-drain electrodes.

Each LED element is formed by stacking, for example, a pixel electrode, an organic layer, and a counter electrode in this order. The organic layer is formed by sequentially stacking a hole injection layer, a hole transport layer, a light-emitting layer, an electron transport layer, and an electron injection layer.

One of the source and drain electrodes of the switching element is electrically connected to the pixel electrode of the LED element, and the other is electrically connected to the address signal line 231.

The data signal line 232 is connected to the gate electrode of the switching element.

While one data signal line 232 electrically connecting to the plurality of the LED packages 27 arranged in a row along the Y-axis direction is shown, three data signal lines 232 are actually provided for respective LED elements of respective colors.

Each of the three data signal lines 232 arranged in the same non-stretching portions 20 and stretching portions 21 may be formed by the same layer and is not electrically connected, or may be arranged in different layers through the interlayer insulating layer.

When the interlayer insulating layer is formed, the slits 22 are formed so as to penetrate an entire flexible wiring substrate including the interlayer insulating layer in the thickness direction.

Similarly, while one data signal line 231 electrically connecting to the plurality of the LED packages 27 arranged in a row along the X-axis direction is shown, three data signal lines 231 are actually provided for respective LED elements of respective colors.

Each of the three data signal lines 231 arranged in the same non-stretching portions 20 and stretching portions 21 may be formed by the same layer and is not electrically connected, or may be arranged in different layers through the interlayer insulating layer.

When the interlayer insulating layer is formed, the slits 22 are formed so as to penetrate an entire flexible wiring substrate including the interlayer insulating layer in the thickness direction.

The slits 22 are provided in the flexible wiring substrate 207 of this embodiment. The operation and effect of the shape of the slits 22 and providing the slits 22 are the same as the flexible wiring substrate 7 of the first embodiment.

In the flexible wiring substrate 207 of this embodiment, by forming the slits 22, similar to the first embodiment, the stretching portions 21 are deformed and stretchable. Thus, the LED display 200 can be deformable three-dimensionally following the free-form curved surface and can be arranged for example, on an object having a cylindrical body, a sphere body, or a complex three-dimensional curved surface along its shape.

Furthermore, for example, if placing displays on four sides of a quadrangular prism, by using the LED display of this embodiment, it is possible to perform image display for four sides in one LED display without providing each display for each side. Since the LED display can be folded to arrange the LED display along the shape of the four sides of the quadrangular prism, it can provide a seamless display. Furthermore, it is possible to reduce the number of parts.

The LED display of this embodiment can also be applied to a display unit of a mobile device such as a notebook personal computer, a portable audio player, a mobile phone, or the like.

Also in this embodiment, similar to the second embodiment, when a force is applied to the flexible wiring substrate 207 upon bending, the stress is easily dispersed in the stretching portions 21 and is less likely to be applied to the non-stretching portions 20 where the electronic components are mounted. As a result, an occurrence of breakage of the electronic components arranged in the non-stretching portions 20 is suppressed, and the LED apparatus with high reliability can be obtained.

In this embodiment, while an example of mounting the LED package on the insulating substrate is described, the switching elements and the LED elements of respective colors on the insulating substrate may be directly formed on the insulating substrate.

In this embodiment, an example in which the LED elements of respective colors of red, blue, and green and the switching elements are arranged for one non-stretching unit 20 is described, but it is not limited thereto. For example, one non-stretching portion 20 may be provided with the LED element of one color and one switching element.

### Fourth Embodiment

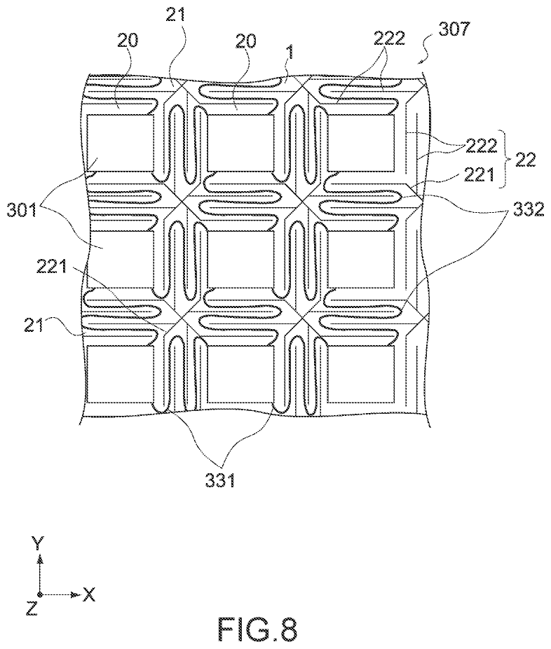

A robot hand as an example of the electronic apparatus including the flexible wiring substrate according to an embodiment of the present technology will be described with reference to FIG. 8.

In order to control an operation such as a grip of the object by the robot hand, it is necessary to detect a gripping state such as sliding of the object and fingertips of the robot hand.

In order to detect the gripping state, it is possible to use a pressure array sensor including a plurality of pressure sensors arranged in an array on the insulating substrate. In this embodiment, the flexible wiring substrate of the present technology is applied to the pressure array sensor. Hereinafter, the pressure array sensor as the flexible wiring substrate will be described.

FIG. 8 is a schematic plan diagram showing a configuration example of the pressure array sensor.

As shown in FIG. 8, a pressure array sensor 307 includes the insulating substrate 1 in which the slits 22 are formed, a plurality of the pressure sensors 301 arranged on the insulating substrate 1, first wiring 331, and second wiring 332.

The robot hand has, for example, a shoulder joint, an upper arm, an elbow joint, a forearm, a wrist, and a hand.

The pressure array sensor 307 is provided, for example, on the palm constituting a part of the hand portion of the robot hand and on the palm surface of each finger.

Pressure values detected by the pressure sensors 301 provided in the pressure array sensor 307, a pressure distribution in a palm and a finger palm surface of each finger can be detected.

The first wiring 331 and the second wiring 332 are arranged intersecting along each of the X-axis direction and the Y-axis direction perpendicular to each other.

The first wiring 331 and the second wiring 332 are arranged in the stretching portions 21 of the insulating substrate 1.

The first wiring 331 and the second wiring 332 are arranged via the insulating layer.

The pressure sensors 301 are provided for each intersection of the first wiring 331 and the second wiring 332. The pressure sensors 301 as the electronic components are arranged in the non-stretching portions 20.

The pressure sensor 301 is a capacitance type pressure sensor for detecting a pressure perpendicular to a sensor surface. The pressure sensor 301 detects the pressure value when the object comes into contact. Based on the detected distribution pressure values, it detects a slip between the object gripped and the fingertips, which is grip information necessary to perform a complicated control of gripping the object by the robot hand.

The pressure sensor 301 has a structure in which a flexible piezoelectric material that is elastically deformed is sandwiched between a first electrode and a second electrode.

The first electrode is electrically connected to the first wiring 331. The second electrode is electrically connected to the second wiring 332. The pressure sensor 301 detects a position where the pressure is applied on the flexible wiring substrate from a capacitance change between the first electrode and the second electrode caused by applying a pressure to the pressure sensor 301.

A detected result of the pressure sensor 301 is output to a controller (not shown) via the first wiring 331 and the second wiring 332, and the grip information is detected by the controller.

The pressure array sensor 307 of this embodiment is also provided with the slits 22 similar to the above-described embodiments. The shapes of the slits 22 and the operation and effect of providing the slits 22 are the same as those of the first embodiment.

In the pressure array sensor 307, by forming the slits 22, similar to the above-described embodiments, the stretching portion 21 is deformed and stretchable. Thus, it is possible to three-dimensionally flexibly deform the pressure array sensor 307.

Therefore, it is possible to locate the pressure array sensor 307 along a non-flat palm and the finger palm surface of each finger of the robot hand and to detect the pressure distribution in the object of the three-dimensional shape with high accuracy.

By using this detection result, it is possible to more appropriately control an operation such as gripping of the object by the robot hand.

In this embodiment, while the pressure sensor is described as an example of the sensor, other sensors such as temperature sensors may be arranged in an array. For example, a temperature distribution can be detected by an array sensor in which the temperature sensors are arranged in the array.

Thus, since the sensor may be the array sensor in which the sensors arranged in the array in the non-stretching portions 20, and the array sensor is three-dimensionally deformable, the array sensor may be arranged at various locations after the array sensor is produced, and sensor arrangement flexibility is high.

A plurality of array sensors can be taken from one wafer collectively by an MEMS (Micro Electro Mechanical System) process. Thus, it is possible to stably obtain high-quality array sensors capable of obtaining high accuracy detection information.

Furthermore, for example, the array sensor may be provided inside an elastic body having a cavity, and an internal pressure of the cavity may be detected. By using such an elastic body, for example, the fingertips of the robot hand is constructed and the internal pressure of the cavity is detected, so that a contact force generated between the fingertips and the object can be detected.

Furthermore, as the insulating substrate, the array sensor may be constituted by using a softer material having excellent flexibility.

<Other Examples of Shapes of Non-Stretching Portions and Shapes of Slits>

The shapes of the non-stretching portions and the shapes of the slits are not limited to the shapes shown in the above embodiments. Hereinafter, other examples will be described as fifth to thirteenth embodiments.

In the following embodiments, the wiring, the electronic components, and the like are not shown. In addition, in the drawings, the regions of the non-stretching portions are shown as being filled with dots. Furthermore, in the drawings, the contours of the non-stretching portions may be indicated by dotted lines.

In each of the following embodiments, the stretching portions are connected to the non-stretching portions, the stretching portions exhibit the stretchability by forming the slits, and the relative positions of the adjacent non-stretching portions can be changed. Thus, a three-dimensionally deformable flexible wiring substrate to which the flexibility and the stretchability are imparted can be provided.

### Fifth Embodiment

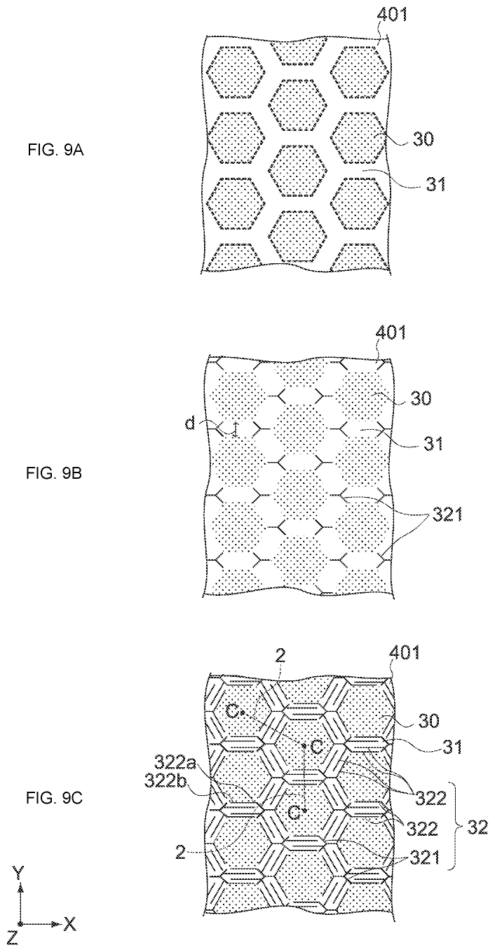

FIG. 9 is a schematic plan diagram of a flexible wiring substrate 401 according to this embodiment. FIG. 9(A) is a diagram for describing the shapes of the non-stretching portions, and does not show the slits. FIG. 9(B) is a diagram for describing the shapes of the first slits, and does not show the second slits. FIG. 9(C) is a diagram for describing the shapes of the first and second slits.

As shown in FIG. 9, the flexible wiring substrate 401 of this embodiment has the plurality of the non-stretching portion 30 and the stretching portions 31 for dividing the non-stretching portions 30.

As in this embodiment, each non-stretching portion 30 may have a substantially regular hexagonal shape. The plurality of the non-stretching portions 30 is arranged in a staggered manner and are spaced apart from each other. By providing the non-stretching portions 30 having the substantially regular hexagonal shapes in the staggered manner, the distances between the adjacent non-stretching portions 30 can be equally spaced over a substrate surface.

As shown in FIG. 9(C), the slits 32 include first slits 321 and second slits 322. The slits 32 are formed by a plurality of linear portions.

As shown in FIG. 9(B), the plurality of the first slits 321 is formed in a substantially Y-shape so as to connect the adjacent non-stretching portions 30.

In this embodiment, in the drawing, the first slits 321 are formed so as to connect the two adjacent non-stretching portions 30 in the longitudinal direction (Y-axis direction) and the two adjacent non-stretching portions 30 in the oblique direction.

Each first slits 321 is formed so that the stretching portion 31 located between the two adjacent non-stretching portions 30 and the stretching portion 31 located between the two other adjacent non-stretching portions 30 are not connected to each other and are separated from each other.

As shown in FIG. 9(C), the plurality, four in this embodiment, of the second slits 322 is formed between the two adjacent non-stretching portions 30, i.e., between the two adjacent first slits 321.

The second slits 322 are non-parallel with an imaginary line 2 connecting between centers C of the two adjacent non-stretching portions 30 arranged through the second slits 322, and are perpendicular to the imaginary line 2 in this embodiment and extended. The second slits 322 are formed such that the two adjacent non-stretching portions 30 are not connected to each other.

Parts of the second slits 322 form contours of substantially regular hexagonal shapes of the non-stretching portions 30 while leaving parts where the non-stretching portions 30 and the stretching portions 31 are connected.

Each linear second slit 322 has one end 322a and the other end 322b. One end 322a is connected to the first slit 321. The other end 322b is not connected to the first slit 321.

Two of the four second slits 322 located between the two adjacent first slits 321 are connected to the first slits 321 of one of the two adjacent first slits 321.

The remaining two second slits 322 are connected to the other first slits 321.

The second slit 322 connected to the one first slit 321 and the second slit 322 connected to the other first slit 321 are alternately formed.

By forming the slits 32 as described above, it is possible to provide the stretching portions 31 having the zigzag belt-shaped bodies, and the stretchability is imparted to the flexible wiring substrate 401.

### Sixth Embodiment

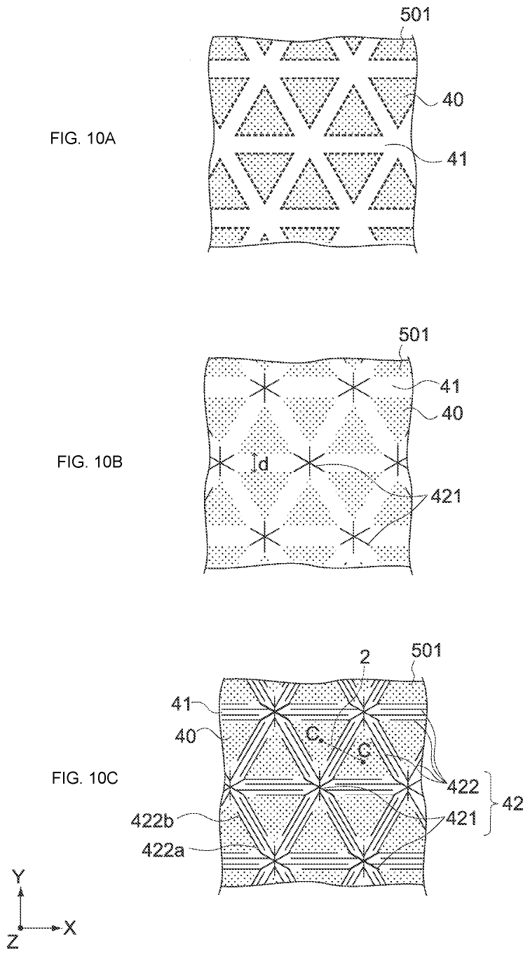

FIG. 10 is a schematic plan diagram of a flexible wiring substrate 501 according to this embodiment. FIG. 10(A) is a diagram for describing the shapes of the non-stretching portions and does not show the slits. FIG. 10(B) is a diagram for describing the shapes of the first slits and does not show the second slits. FIG. 10(C) is a diagram for describing the shapes of the first and second slits.

As shown in FIG. 10, the flexible wiring substrate 501 of this embodiment includes a plurality of non-stretching portions 40 and a stretching portions 41 for dividing the non-stretching portions 40.

As shown in FIG. 10, each non-stretching portion 40 may have an equilateral triangular shape. When one side of the plurality of the non-stretching portions 40 is arranged along the X-axis direction, a triangular non-stretching portion 40 whose opposite apex is positioned above the one side and an inverted triangular non-stretching portion 40 whose opposite vertex is positioned below the one side are alternately arranged along the X-axis direction and the Y-axis direction.

As shown in FIG. 10(C), the slits 42 include first slits 421 and second slits 422. The slits 42 are configured of a plurality of linear portions.

As shown in FIG. 10(B), the first slits 421 are formed so as to connect the two adjacent non-stretching portions 40. In this embodiment, in the drawing, the first slits 421 are formed in a center of a regular hexagonal assembly as an entire shape formed by assembling six equilateral triangular non-stretching portions 40. The first slit 421 has a shape in which one linear line connecting the two adjacent non-stretching portions 40 along the Y-axis direction among the six non-stretching portions 40 forming the assembly and two linear lines intersecting with the one linear line and connecting the two adjacent non-stretching portions along two sets of oblique directions for each set.

In this manner, the first slits 421 are formed so that the stretching portion 41 located between the two adjacent non-stretching portions 40 and the stretching portion 41 located between the other adjacent non-stretching portion 40 are not connected to each other and are separated from each other.

As shown in FIG. 10(C), a plurality, four in this embodiment, of the second slits 422, is formed between the two adjacent non-stretching portions 40, i.e., between the two adjacent first slits 412 are formed.

The second slits 422 extend in non-parallel with the imaginary line 2 connecting between the centers C of the adjacent two non-stretching portions 40 arranged through the second slit 422 and perpendicular to the imaginary line 2 in this embodiment. The second slits 422 are formed so that the two adjacent non-stretching portions 40 are not connected to each other.

Parts of the second slits 422 are formed so as to form contours of substantially equilateral triangles of the non-stretching portions 40 while leaving parts where the non-stretching portions 40 and the stretching portions 41 are connected.

The second slit 422 has one end 422a and the other end 422b. One end 422a is connected to the first slit 421. The other end 422b is not connected to the first slit 421.

Two of the four second slits 422 located between the two adjacent first slit 421 are connected to the first slits 421 of one of the two adjacent first slits 421.

The remaining two second slits 422 are connected to the other first slits 421.

The second slit 422 connected to the one first slit 421 and the second slit 422 connected to the other first slit 421 are alternately formed.

By forming the slits 42, it is possible to provide the stretching portions 41 having the zigzag belt-shaped bodies.

### Seventh Embodiment

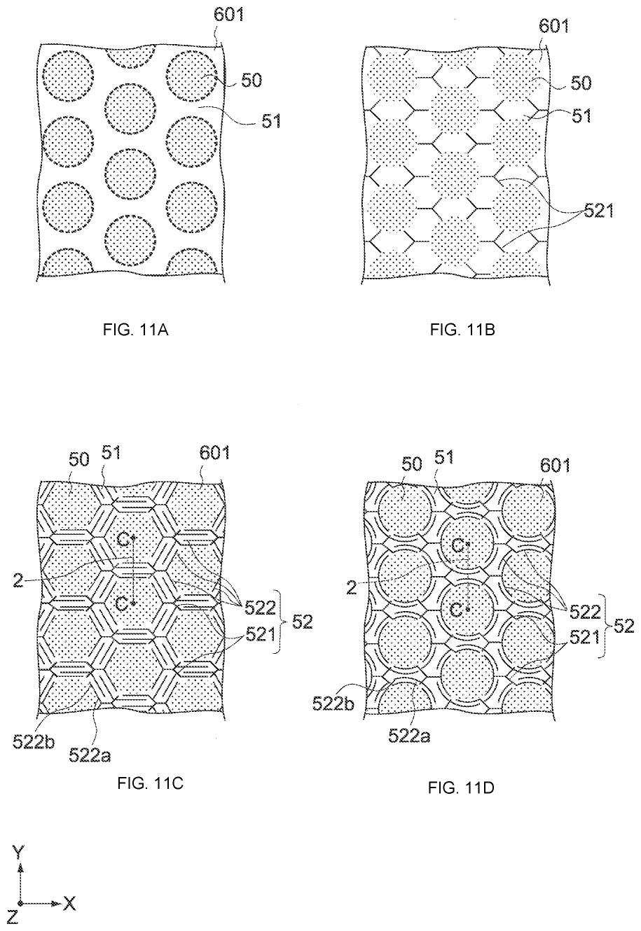

FIG. 11 is a schematic plan diagram of a flexible wiring substrate 601 according to this embodiment. FIG. 11(A) is a diagram for describing the shapes of the non-stretching portions and does not show the slits. FIG. 11(B) is a diagram for describing the shapes of the first slits and does not show the second slits. FIG. 11(C) is a diagram for describing the shapes of the first and second slits. FIG. 11(D) is a diagram for describing other shapes of the first and second slits.

As shown in FIG. 11, the flexible wiring substrate 601 of this embodiment includes a plurality of non-stretching portions 50 and stretching portions 51 for dividing the non-stretching portions 50.

As in this embodiment, each of the non-stretching portions 50 may have a substantially circular shape. The plurality of the non-stretching portions 50 having circular shapes is arranged in a staggered manner.

Slits 52 are formed in the stretching portions 51. The slit 52 has a first slit 521 and a second slit 522.

Incidentally, the slits shown in FIGS. 11(C) and (D) are the same shapes as to the first slits but different shapes as to the second slits. While the second slits shown in FIG. 11(C) are linear, the second slits shown in FIG. 11(D) are arcuate.

As shown in FIG. 11(B), the first slits 521 are formed in substantially Y-shapes so as to connect the two adjacent non-stretching portions 50.

Each of the first slits 521 is formed so that the stretching portion 51 located between the two adjacent non-stretching portions 50 and the stretching portion 51 located between the other two adjacent non-stretching portions 50 are separated from each other.

As shown in FIG. 11(C), a plurality, four in this embodiment, of the linear second slits 522, is formed between the two adjacent non-stretching portions 50 and between the two adjacent first slits 521.

The second slits 522 extend in non-parallel with the imaginary line 2 connecting between the centers C of the adjacent two non-stretching portions 50 and perpendicular to the imaginary line 2 in this embodiment. The second slits 522 are formed so that the two adjacent non-stretching portions 50 are not connected to each other.

In FIG. 11(C), since the second slit 522 is linear, a contour of the non-stretching portion 50 becomes a substantially regular hexagonal shape, which is the same slit shape as the fifth embodiment described above, but here, the substantially regular hexagonal shape is regarded as a substantially circular shape.

The second slit 522 has one end 522a and the other end 522b. One end 522a is connected to the first slit 521. The other end 522b is not connected to the first slit 521.

Parts of the second slits 522 are formed so as to form circular contours of the non-stretching portions 50 having substantially circular shapes while leaving parts where the non-stretching portions 50 and the stretching portions 51 are connected to each other.

Between the two adjacent first slits 521, the second slit 522 connected to the one first slit 521 of the two first slits 521 and the second slit 522 connected to the other first slit 52 are alternately formed.

By forming the slits 52, it is possible to provide the stretching portions 51 having the zigzag belt-shaped bodies.

As shown in FIG. 11(D), the second slit 522 may not be linear but may have an arc shape. The second slits 522 are non-parallel with the imaginary line 2 connecting between the centers C of the two adjacent non-stretching portions 50. The shapes of the non-stretching portions 50 are substantially circular.

As in the above embodiments, the shapes and the arrangements of the non-stretching portions can be of various forms.

### Eighth Embodiment

In the first to fourth embodiments in which the shapes of the non-stretching portions are square shapes, the shapes of the first slits are X-shapes, but it is not limited thereto.

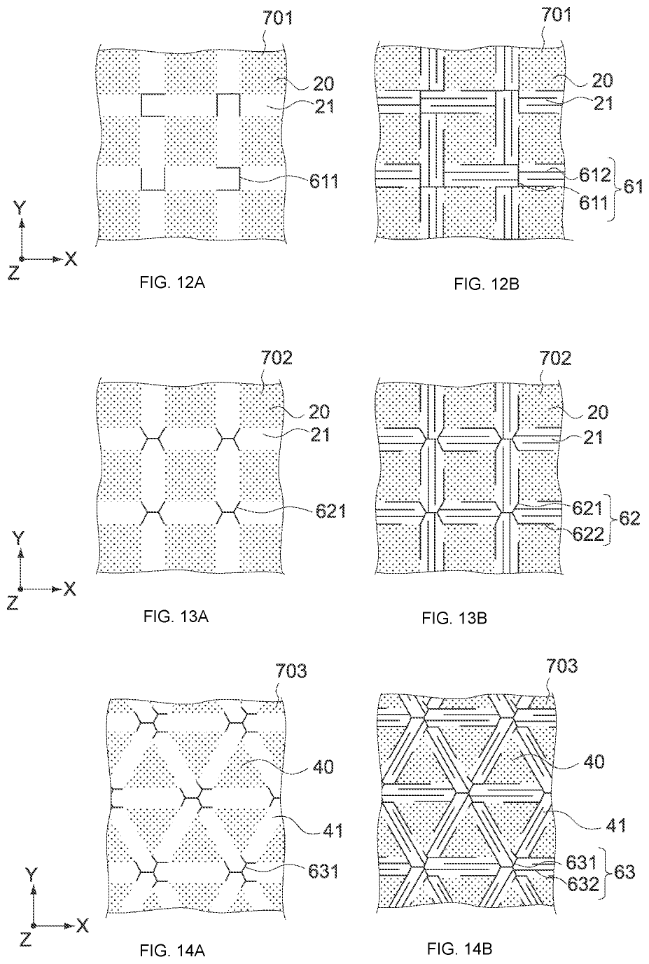

For example, as shown in FIG. 12(A), substantially U-shaped first slits 611 formed from linear lines may be used.

FIG. 12 is a schematic plan diagram of a flexible wiring substrate 701 according to this embodiment. FIG. 12(A) is a diagram for describing the shapes of the first slits. FIG. 12(B) is a diagram for describing the shapes of the first and second slits.

As shown in FIG. 12, even as the substantially U-shaped first slits 611, each non-stretching portion 20 can be formed such that the relative positions can be changed independently of each other. Each divided region formed by dividing the stretching portions 21 with the first slits 611 is connected only to the two non-stretching portions 20 facing arranged through the divided region therebetween.

In contrast to the U-shaped first slits 611, as shown in FIG. 12(B), by forming a plurality of second slits 612 in which each one end is connected to the first slit 611 and the other end is not connected to the first slit 611 is formed, it is possible to three-dimensionally deform the flexible wiring substrate 701.

### Ninth Embodiment

In the first to fourth embodiments in which the shapes of the non-stretching portions are the square shapes, the shapes of the first slits are X-shapes, but it is not limited thereto.

For example, as shown in FIG. 13(A), in a case where the shapes and positions of the non-stretching portions 20 and the stretching portions 21 are set so that the regions where intersections of the regions where lattice-shaped stretching portions 21 are formed are squares, first slits 621 may be formed along shapes of shortest split lines where a total sum of lengths of line segments connecting the four vertices of the squares is minimized.

FIG. 13 is a schematic plan diagram of a flexible wiring substrate 702 according to this embodiment. FIG. 13(A) is a diagram for describing the shapes of the first slits 621. FIG. 13(B) is a diagram for describing the shapes of the slits 62 having the first slits 621 and the second slits 622.

As shown in FIG. 13(A), the first slits 621 may be formed along the shapes of the shortest split lines, and the cut length can be shortened. Therefore, when forming the slits by laser processing, it is possible to shorten a slit formation time, and to improve the yield.

In contrast to the first slits 621 of the shapes of the shortest split lines, as shown in FIG. 13(B), second slits 622 in which each one end is connected to the first slits 621 and the other end is not connected to the first slits 621 to from the slits 62, thereby three-dimensionally deforming the flexible wiring substrate 702.

Note that, the case where the regions forming the first slits are square is taken as an example, but other shapes may be taken, and by providing the first slits along the shapes of the shortest split lines in the shape, it is possible to shorten the cut length.

### Tenth Embodiment

The shapes of the first slits are not limited to the shapes shown in the sixth embodiment in a case where the shapes of the non-stretching portions are equilateral triangle.

FIG. 14 is a schematic plan diagram of a flexible wiring substrate 703 according to this embodiment. FIG. 14(A) is a diagram for describing shapes of first slits 631. FIG. 14(B) is a diagram for describing shapes of slits 63 having the first slits 631 and second slits 632.

The first slits 631 having the shapes as shown in FIG. 14(A) may be formed.

In contrast to the first slits 631 having such shapes, as shown in FIG. 14(B), by forming the second slits 632 in which each one end is connected to the first slit 631 and the other end is not connected to the first slit 631 to form the slits 63, the flexible wiring substrate 703 can be three-dimensionally deformable.

As in the eighth to tenth embodiments described above, the shapes of the first slits may take various shapes. In addition, it is not limited to the shapes described herein.

### Eleventh Embodiment

In the first to fourth embodiments in which the shapes of the non-stretching portions are the square shapes, while arranging the non-stretching portions in a matrix as an example, but it is not limited thereto.

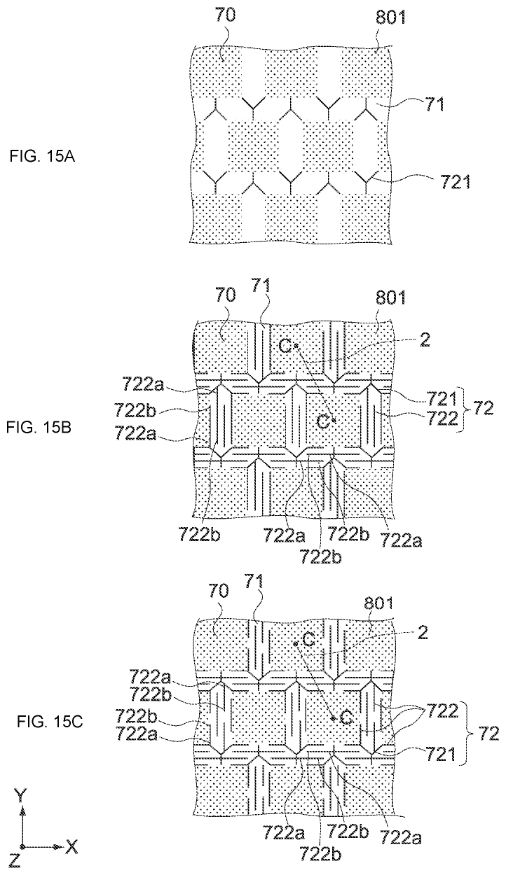

FIG. 15 is a schematic plan diagram of a flexible wiring substrate 801 according to this embodiment.

FIG. 15(A) is a diagram for describing shapes of the first slits.

FIG. 15(B) is a diagram for describing the shapes of the first and second slits.

FIG. 15(C) is a diagram for describing other shapes of the first and second slits.

The slits shown in FIGS. 15(B) and (C) are the same shapes as to the first slits but different shapes as to the second slits.

As shown in FIG. 15, the flexible wiring substrate 801 has a plurality of non-stretching portions 70, and stretching portions 71 for dividing the non-stretching portions 70.

As shown in FIG. 15, the plurality of square-shaped non-stretching portions 70 may be arranged in a staggered manner spaced apart from each other.

The slits 72 forming the stretching portions 71 have first slits 721 and second slits 722.

As shown in FIG. 15(A), a plurality of the first slits 721 is formed in Y-shapes and inverted Y-shapes so as to connect the two adjacent non-stretching portions 70. In the drawing, the first slits 721 are formed at a center of an assembly of generally triangle or generally inverse triangle in an entire shape formed by assembling the three adjacent non-stretching portions 70. Each first slit 721 is formed so as to connect the three non-stretching portions 70 forming the assembly.

Each first slit 721 is formed so that the stretching portion 71 located between the two adjacent non-stretching portions 70 and the stretching portion 71 located between the other two adjacent non-stretching portions 70 are separated from each other.

As shown in FIG. 15(B), four second slits 722 are formed between the two adjacent non-stretching portions 70 and between the two adjacent first slits 721.

The second slit 722 extends in non-parallel with the imaginary line 2 connecting between the centers C of the two adjacent non-stretching portions 70. The second slit 722 is formed so that the two adjacent non-stretching portions 70 are not connected to each other.

Each second slit 722 has one end 722a and the other end 722b. The one end 722a is connected to the first slit 721. The other end 722b is not connected to the first slit 721.

Between the two adjacent first slits 721, the second slit 722 connected to the one first slit 721 of the two first slits 721 and the second slit 722 connected to the other first slit 721 are alternately formed.

By forming the slits 72, the stretching portions 71 can be formed as narrow band-like bodies.

In addition, the shapes of the second slits 722 are not limited to the shapes shown in FIG. 15(B), and may be the shapes shown in FIG. 15(C).

As shown in FIG. 15(C), there may be the second slits 722 in which both ends are not connected to the first slits 721 among a plurality of the second slits 722.

In a pattern of the second slits 722 shown in FIG. 15(C), as compared with a pattern shown in FIG. 15(B), the stretchability in the X-axis direction becomes small. Thus, by changing the slit patterns, an amount of stretching may be changed as appropriate in different directions from each other on the surface of the flexible wiring substrate. Furthermore, on the surface of the slit wiring substrate, by changing the extending directions of the slits, or by changing the slit pattern for each different area, the amount of stretching of the flexible wiring substrate may be changed on the surface as appropriate.

### Twelfth Embodiment

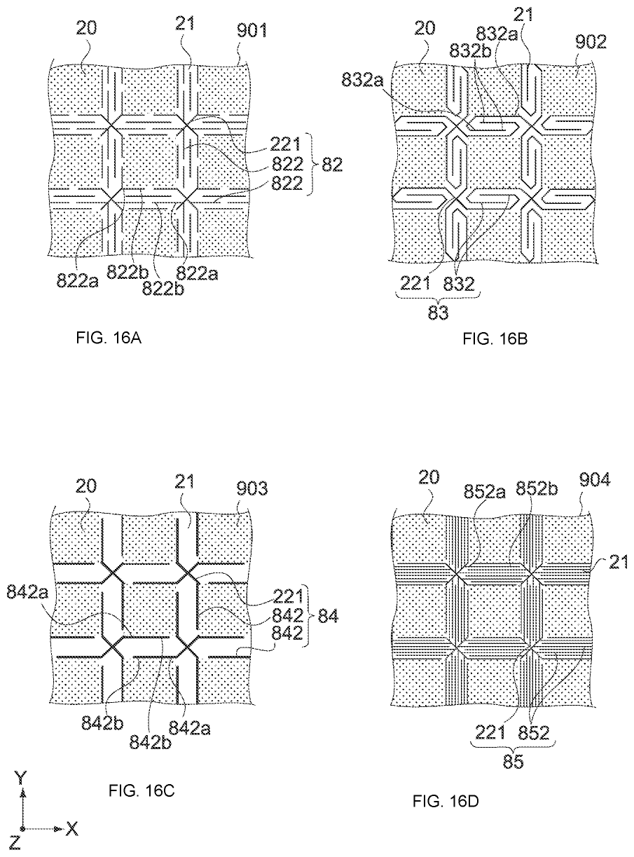

With reference to FIG. 16, a modified embodiment of the shapes of the slits will be described. Each diagram of FIGS. 16(A)-(D) is a schematic plan diagram of the flexible wiring substrate.

A flexible wiring substrate 901 shown in FIG. 16(A) differs from the first embodiment only in that the shapes of the second slits are different.

In the flexible wiring substrate 901, slits 82 having first slits 221 and second slits 822 are formed.

As shown in FIG. 16(A), there may be the second slits 822 in which both ends are not connected to the first slit 221 among a plurality of the second slits 822.

In the example shown in the drawing, the second slits 822 in which each one end 822a is connected to the first slit 221 and the other end 822b is connected to the second slit 222 are also formed.

Part of the second slits 822 are formed so as to form substantially square shape contours of the non-stretching portions 20 while leaving parts where the non-stretching portions 20 and the stretching portions 21 are connected.

A flexible wiring substrate 902 shown in FIG. 16(B) differs from the first embodiment only in that the shapes of the second slits are different.

In the flexible wiring substrate 902, slits 83 having first slits 221 and second slits 832 are formed.

As shown in FIG. 16(B), the second slit 832 may be a substantially U-shaped having a folded portion formed by four linear portions are continuous.

In this embodiment, the two second slits 832 are formed between the two adjacent non-stretching portions 20 and between the two adjacent first slits 221. Two linear portions of the four linear portions forming one second slit 832 are formed parallel to each other, and by the two second slits 832, the four linear portions are positioned between the two adjacent non-stretching portions 20.

By forming the slits 83, it is possible to provide the stretching portions 21 having the belt-shaped bodies.

Between the two adjacent first slits 221, the second slit 832 connected to the one first slit 221 of the two first slits 221 and the second slit 832 connected to the other first slit 221 are alternately formed.

One end 832a of the second slit 832 is connected to the first slit 221, and the other end 832b is not connected to the first slit 221.

The second slits 832 are formed so as to form substantially square shape contours of the non-stretching portions 20 while leaving parts where the non-stretching portions 20 and the stretching portions 21 are connected.

A flexible wiring substrate 903 shown in FIG. 16(C) differs from the first embodiment only in that the number of the second slits is different.

In the flexible wiring substrate 903, slits 84 having first slits 221 and second slits 842 are formed.

In the first embodiment, an example in which the four linear second slits 222 are formed between the two adjacent first slits 221 is described, but the number of the second slits 222 is not limited thereto, and may be, for example, two, as shown in FIG. 16(C).

The second slits 842 are formed so as to form substantially square shape contours of the non-stretching portions 20 while leaving parts where the non-stretching portions 20 and the stretching portions 21 are connected.

Between the two adjacent first slits 221, the second slit 842 connected to the one first slit 221 of the two first slits 221 and the second slit 842 connected to the other first slit 221 are alternately formed.

One end 842a of the second slit 842 is connected to the first slit 221, and the other end 842b is not connected to the first slit 221.

By forming the slits 84, it is possible to provide the stretching portions 21 having the belt-shaped bodies.

A flexible wiring substrate 904 shown in FIG. 16(D) differs from the first embodiment only in that the number of the second slits is different.

In the flexible wiring substrate 904, slits 85 having first slits 221 and second slits 852 are formed.

In the first embodiment, an example in which the four linear second slits 222 are formed between the two adjacent first slits 221 is described, but the number of the second slits 222 is not limited thereto, and may be, for example, eight, as shown in FIG. 16(D).

Of the eight linear second slits 852 formed in a region sandwiched between the two adjacent first slits 221, the four second slits 852 are connected to one of the first slits 221.

The other four second slits 852 are connected to the other first slit 221.

The four second slits 852 connected to the one first slit 221 and the four second slits 852 connected to the other first slit 221 are alternately formed.

Parts of the second slits 852 are formed so as to form substantially square shape contours of the non-stretching portions 20 while leaving parts where the non-stretching portions 20 and the stretching portions 21 are connected.

By forming the slits 85, it is possible to provide the stretching portions 21 having the belt-shaped bodies.

As in the above embodiments, the number of slits, the position of the slits, symmetry of the slit positions, the position of the connection between the non-stretching portions and the stretching portions, etc. can be changed.

As described above, the number of the second slits can be appropriately set. It is possible to adjust a degree of the stretchability imparted to the flexible wiring substrate by adjusting the number of the second slits. For example, the stretchability can be increased by increasing the number of the second slits.

In the region sandwiched by the two first slits adjacent, it is desirable that at least the two second slits not located on the same linear line are formed. Then, one end of each of the two second slits is connected to the first slit and the other end is not connected to the first slit. One of the two second slits is connected to the first slit of one of the two adjacent first slits and the other second slit is connected to the other of the first slit.

By forming such a slit, it is possible to provide the stretching portions having stretchable shapes and to provide the flexible wiring substrate in which the relative positions of the adjacent non-stretching portions can be changed. By increasing the number of the second slits, the stretchability of the stretching portion can be increased.

### Thirteenth Embodiment

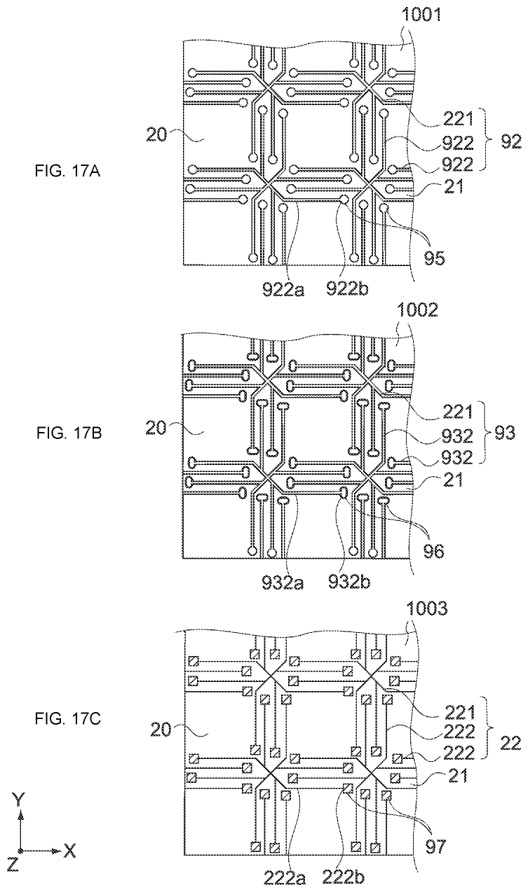

With reference to FIG. 17, a modified embodiment will be described. Each diagram of FIGS. 17(A)-(C) is a schematic plan diagram of the flexible wiring substrate.

A flexible wiring substrate 1001 shown in FIG. 17(A) differs from the first embodiment only in that the second slits have widened portions at the other ends.

In the flexible wiring substrate 1001, slits 92 having first slits 221 and second slits 922 are formed.

Each other end 922b of the second slits 922 is cut into a regular circular shape, and the other portions are cut into linear shapes. By cutting each other end 922b into the regular circular shape, a widened portion 95 wider than a slit width of a portion linearly cut is formed.

While in each of the embodiments described above, the slit is shown by one solid line, in order to more easily understand the shape of the widened portion 95, FIG. 17(A) shows so that there are gaps in the portions linearly cut.

By providing the circular widening portion 95, a stress applied to the tip of the second slit 922 (corresponding to the other end 922b) can be dispersed by the stretching of the stretching portion 21, and cracks are suppressed from entering the flexible wiring substrate 1001 from the tip of the second slit 922 as a starting point.

Each second slit 922 having such a circular widening portion 95, for example, can be formed by changing a processing line width only at the tip of the second slit 922 thicker at the time of laser processing.

Changing the processing line width can be realized by changing a focus of laser and decreasing a resolution. Alternatively, the processing line width may be changed using aperture masks having different opening diameters. In either case, by changing the processing line width in the same laser processing machine, it is possible to form the second slits 922 having the widened portions including linear portions and circular portions.

Thus, a pattern itself of the slits to be processed by the laser processing machine can be formed only by the linear lines, and it is possible to form the circular widened portions by changing the processing line width.

Incidentally, the shape of the widened portion is not limited to circular and may be, for example, elliptical.

A flexible wiring substrate 1002 shown in FIG. 17(B) differs from the embodiment shown in FIG. 17(A) only in that shape of the widened portion is different.

In the flexible wiring substrate 1002, slits 93 having first slits 221 and second slits 932 are formed.

Each other end 932b of the second slits 932 is cut into an elliptical shape, and the other portions are cut into linear shapes. By cutting each other end 932b into the elliptical shape, a widened portion 96 wider than a slit width of a portion linearly cut is formed at the other end 932b of the second slits 932.

Similar to FIG. 17(A), in order to more easily understand the shape of the widened portion 96, FIG. 17(B) shows so that there are gaps in the portions linearly cut.

By providing the elliptical widening portion 96, a stress applied to the tip of the second slit 932 (corresponding to the other end 932b) can be dispersed by the stretching of the stretching portion 21, and cracks are suppressed from entering the flexible wiring substrate 1002 from the tip of the second slit 932 as a starting point.

Such an elliptical widening portion 96 can be formed so as to connect the two circular widened portions of described in FIG. 17(A) by laser processing.

In addition, each reinforcing portion 97 may be provided as shown in FIG. 17(C) without providing the widening portion.

A flexible wiring substrate 1003 shown in FIG. 17(C) differs from the embodiment shown in FIG. 17(A) only in that the reinforcing portions 97 are further provided on the other ends 222b of the second slits 222.

Each reinforcing portion 97 is provided so as to cover each other end 222b of the second slits 222. The material used for the reinforcing portions 97 is not particularly limited, and for example, a metal can be used. By providing the reinforcing portions 97, strength of each tip (corresponding to the other end 222b) of the second slits 222 can be improved, and cracks are suppressed from entering the flexible wiring substrate 1003 from the tip of the second slit 222 as a starting point by the stretching of the stretching portions 21.

As described with reference to FIGS. 17(A)-(C), providing the widened portion at the tip of the second slit, or by providing the reinforcing portion, cracks are suppressed from entering the flexible wiring substrate from the tip of the second slit 932 as a starting point.

Therefore, it is possible to suppress the occurrence of disconnection of the wiring arranged in the stretching portions 21, and to obtain a highly reliable flexible wiring substrate and an electronic apparatus using the same with disconnection failure of the wiring being reduced.

As described above, in the present technology, by forming the slits, it is possible to obtain the three-dimensionally deformable flexible wiring substrate.

Embodiments of the present technology are not limited to the above-mentioned embodiments, and various modifications can be made without departing from the gist of the present technology.

For example, each of the above-described embodiments may be appropriately combined, and the LED display described in the third embodiment may be used for the display unit of the heartbeat measuring apparatus described in the second embodiment. Furthermore, the touch panel described in the fourth embodiment may be provided on the LED display to enable touch inputs.

In the above-described embodiments, the heart rate measuring apparatus, the LED display, and the robot hand are described as examples of the electronic apparatus using the flexible wiring substrate, it is not limited thereto.

For example, the flexible wiring substrate can be applied to an electrode for living body for detecting a potential fluctuation from a living body such as an electrocardiogram, a brain wave, or an electromyograph. The electrode for living body is used by being attached to a body surface. As the electrode for living body, the flexible wiring substrate made of a metal sheet having the stretching portions and the non-stretching portions in which the slits are formed, or the flexible wiring substrate in which the slits are formed in a composite body to which the metal sheet is attached on a support member can be used.

As another example, the flexible wiring substrate according to the present technology can be applied to a touch panel such as a resistive film type or a capacitive type.

Furthermore, it may be the flexible wiring substrate in which a plurality of solar cells is arranged in the non-stretching portions as light receiving elements.

Furthermore, the flexible wiring substrate in which a plurality of cell batteries is arranged in the non-stretching portions may be provided as a battery having the flexibility and the stretchability.

Furthermore, it may be flexible wiring substrate in which a plurality of [00s is placed in the non-stretching portions and wiring for electrically connecting respective capacitors is arranged in the stretching portions, which efficiently performs charging of the respective capacitors.

In addition, the flexible printed circuit substrate according to the present technology is applicable to any electronic apparatus including any Personal Digital Assistant (PDA) such as a smart phone and a tablet terminal, a medical device, a gaming device, or a home appliance.

The above-described embodiments exemplify a uniform slit pattern in which the same slit shape is repeated on a surface, but it is not limited thereto.

For example, the sizes of the plurality of the non-stretching regions may be different in the substrate surface, or the slit patterns may be partially different in the substrate surface.

By making the sizes of the non-stretching regions and the slit pattern partially different on the surface, the stretchability can be made different on the surface. Thus, depending on the shape required for the electronic apparatus to which the flexible wiring substrate is applied, it is possible to vary the stretchability of the flexible wiring substrate on the substrate surface.

The above-described embodiments exemplify that the laser processing machine is used for forming the slits, but a cutting plotter may be used.

The present technology may also have the following structures.

(1)

A flexible wiring substrate, including:

a plurality of non-stretching portions spaced apart from each other; and

stretching portions that divide the plurality of the non-stretching portions, connect the adjacent non-stretching portions, and are stretched and contracted by formed slits to be capable of changing relative positions between the non-stretching portions.

(2)

The flexible wiring substrate according to (1), in which

the slits include first slits that separate the stretching portion located between two adjacent non-stretching portions from the stretching portion located between two other adjacent non-stretching portions.

(3)

The flexible wiring substrate according to (2), in which

the slits include second slits each extending in non-parallel with an imaginary line connecting respective centers of the two adjacent non-stretching portions in the stretching portions located between the two adjacent non-stretching portions.

(4)

The flexible wiring substrate according to (3), in which

the second slits are perpendicular to the imaginary line.

(5)

The flexible wiring substrate according to (3) or (4), in which

one end of the second slit is connected to the first slit, and the other end is not connected to the first slit.

(6)

The flexible wiring substrate according to any one of (3)

to (5), in which

a plurality of the first slits is formed at the stretching portions,

at least the two second slits are formed between the two adjacent first slits,

one end of the second slit of the two second slits is connected to one of the two adjacent first slits, and

one end of the other second slit is connected to the other first slit.

(7)

The flexible wiring substrate according to any one of (3) to (6), in which

the second slits have widening portions at the other ends.

(8)

The flexible wiring substrate according to any one of (3) to (6), further including:

reinforcing portions covering the other ends of the second slits.

(9)

The flexible wiring substrate according to any one of (1) to (8), in which

the slits have shapes extending in different directions.

(10)

The flexible wiring substrate according to any one of (1) to (9), in which

the slits are configured of linear portions.

(11)

The flexible wiring substrate according to any one of (1) to (10), further including:

electronic components provided in the non-stretching portions, and

wiring provided in the stretching portions for electrically connecting the electronic components.

(12)

The flexible wiring substrate according to any one of (1) to (10), in which

the flexible wiring substrate is made of a conductive member.

(13)

An electronic apparatus, including:

flexible wiring substrate including a plurality of non-stretching portions spaced apart from each other, and stretching portions that divide the plurality of the non-stretching portions, connect the adjacent non-stretching portions, and are stretched and contracted by formed slits to be capable of changing relative positions between the non-stretching portions.

### REFERENCE SIGNS LIST

1 insulating substrate
2 imaginary line
7, 17, 207, 401, 501, 601, 701 to 703, 801, 901 to 904, 1001 to 1003 flexible wiring substrate
14 acceleration sensor (electronic component)
20, 20a to 20i, 30, 40, 50, 70 non-stretching portion
21, 21a, 31, 41, 51, 71 stretching portion
22, 32, 42, 52, 61 to 63, 72, 82 to 85, 92, 93 slit
27 LED package (electronic component)
33 to 38 wiring
95, 96 widened portion
97 reinforcing portion
100 heartbeat measuring apparatus (electronic component)
121 first LED (electronic component)
122 first PD (electronic component)
131 second LED (electronic component)
132 second PD (electronic component)
151 IC chip (electronic component)
200 LED display (electronic component)
221, 221a to 221d, 321, 421, 521, 611, 621, 631, 721 first slit
222, 322, 422, 522, 612, 622, 632, 722, 822, 832, 842, 852, 922, 932 one end of second slit (one end)
222a, 322a, 422a, 522a, 722a, 822a, 832a, 842a, 852a, 922a, 932a other end of second slit (other end)
231 address signal line (wiring)
232 data signal line (wiring)
301 pressure sensor (electronic component)
307 pressure array sensor (flexible wiring substrate)
331 first wiring (wiring)
332 second wiring (wiring)
C center

1. A flexible wiring substrate, comprising:
a plurality of non-stretching portions spaced apart from each other; and
stretching portions that divide the plurality of the non-stretching portions, connect the adjacent non-stretching portions, and are stretched and contracted by formed slits to be capable of changing relative positions between the non-stretching portions.

2. The flexible wiring substrate according to claim 1, wherein
the slits include first slits that separate the stretching portion located between two adjacent non-stretching portions from the stretching portion located between two other adjacent non-stretching portions.

3. The flexible wiring substrate according to claim 2, wherein
the slits include second slits each extending in non-parallel with an imaginary line connecting respective centers of the two adjacent non-stretching portions between the two adjacent non-stretching portions.

4. The flexible wiring substrate according to claim 3, wherein
the second slits are perpendicular to the imaginary line.

5. The flexible wiring substrate according to claim 4, wherein
one end of the second slit is connected to the first slit, and the other end is not connected to the first slit.

6. The flexible wiring substrate according to claim 5, wherein
a plurality of the first slits is formed at the stretching portions,
at least the two second slits are formed between the two adjacent first slits,
one end of one second slit of the two second slits is connected to one of the two adjacent first slits, and
one end of the other second slit is connected to the other first slit.

7. The flexible wiring substrate according to claim 3, wherein
the second slits have widening portions at the other ends.

8. The flexible wiring substrate according to claim 3, further comprising:
reinforcing portions covering the other ends of the second slits.

9. The flexible wiring substrate according to claim 7, wherein
the slits have shapes extending in different directions.

10. The flexible wiring substrate according to claim 9, wherein
the slits are configured of linear portions.

11. The flexible wiring substrate according to claim 3, further comprising:
electronic components provided in the non-stretching portions, and
wiring provided in the stretching portions for electrically connecting the electronic components.

12. The flexible wiring substrate according to claim 3, wherein
the flexible wiring substrate is made of a conductive member.

13. An electronic apparatus, comprising:
flexible wiring substrate including a plurality of non-stretching portions spaced apart from each other, and stretching portions that divide the plurality of the non-stretching portions, connect adjacent non-stretching portions, and are stretched and contracted by formed slits to be capable of changing relative positions between the non-stretching portions.
